(19) United States
(12) Patent Application Publication
(10) Pub. No.: US 2023/0183774 A1
(43) Pub. Date: Jun. 15, 2023

(54) COLORIMETRIC ASSAY FOR HIGH THROUGHPUT, FACILE AND RAPID ANTIMICROBIAL SUSCEPTIBILITIES TESTING

(71) Applicant: Georgia Tech Research Corporation, Atlanta, GA (US)

(72) Inventors: Robert Martin Dickson, Atlanta, GA (US); Alexandra B. Filbrun, Atlanta, GA (US); Prakash C. Khanal, Atlanta, GA (US)

(21) Appl. No.: 18/064,511

(22) Filed: Dec. 12, 2022

Related U.S. Application Data
(60) Provisional application No. 63/288,196, filed on Dec. 10, 2021.

(51) Int. Cl. C12Q 1/20; G01N 21/78; B01L 3/00; C12Q 1/04

(52) U.S. Cl. CPC C12Q 1/20; G01N 21/78; B01L 3/5085; C12Q 1/04; B01L 2300/0829; B01L 2300/0654

(57) ABSTRACT

An exemplary embodiment of the present disclosure provides a system for detecting antimicrobial resistance of a bacteria in a biological sample. In some embodiments, the system can include a plurality of containers, a detecting agent in each of the plurality of containers, and an antimicrobial agent in at least a portion of the plurality of containers. The antimicrobial agent is disposed in at least one of the plurality of containers. Each of the containers can contain at least a portion of the biological sample. The detecting agent can be configured to produce optically detectable changes responsive to bacterial respiration or growth, directly from patient samples of from patient sample cultures.

### CROSS-REFERENCE TO RELATED APPLICATIONS

This application claims the benefit of U.S. Provisional Application Ser. No. 63/288,196, filed on 10 Dec. 2021, which is incorporated herein by reference in its entirety as if fully set forth below.

### FIELD OF THE DISCLOSURE

The various embodiments of the present disclosure relate generally to systems and methods for testing for antimicrobial resistance, and more particularly to rapid antibiotic susceptibility test directly from bodily fluids and cultures.

### BACKGROUND

Antimicrobial resistant (AMR) infections are rising at an alarming rate due, in part, to lack of rapid susceptibility testing. The lack of rapid diagnostics often prompts clinicians to administer broad-spectrum antibiotics to knock down potential bacterial infections. Such antibiotic overuse is a major contributor to increased bacterial resistance towards existing antibiotics.

AMR also has a large economic impact, as it leads to prolonged hospital stays and increases healthcare costs by ˜$35 billion per year in the US alone. Low- and medium-income countries are even more acutely affected by the increase in AMR and the costs associated with it.

While bacterial infections are problematic in multiple bodily fluids, bloodstream infections (BSIs) are a leading cause of mortality and morbidity globally and are predominantly a result of very small numbers of bacteria surviving in the blood stream. It has been reported that BSIs lead to 6 million deaths and affect 30 million people annually. The high BSI-related death toll is largely attributed to the lack of rapid diagnostics that mandates empirical and unsuitable treatment. While rapid administration of empiric antibiotics improves any individual patient outcome, these broad treatments increase future AMR infections if the bacteria present are resistant to the drug administered, thereby potentially increasing mortality overall. This underscores the urgency to develop rapid antimicrobial susceptibility tests (ASTs) for the treatment of patients to ensure their survival and reduce associated healthcare costs. An ideal AST should be rapid, cost-effective, and easily implemented, even in low resource settings, to reduce the rising concerns of deaths and economic burden caused by BSIs world-wide.

Blood cultures are still the necessary first step in gold standard diagnosis of BSIs and sepsis. However, one major limitation is that the current susceptibility testing timelines exceed 50 hours from initial blood draw when including 24 hours for the blood culture to turn positive, as they require additional subculturing and isolation steps prior to susceptibility determination. Standard broth microdilution (BMD) requires more than 60 hours and the instrumentation intensive Vitek2 analysis requires about 54.5 hours from initial blood draw. This long AST timeline not only leads to poor patient outcomes, but also contributes to incidence of AMR.

Therefore, there is a need for a fast, simple and easy to use antibiotic susceptibility tests and methods to test for antimicrobial resistant infections using bodily fluids and cultures.

### BRIEF SUMMARY

The present disclosure relates to systems and methods for detecting antimicrobial resistance in a sample. An exemplary embodiment of the present disclosure provides a system for detecting antimicrobial susceptibility of a bacteria in a biological sample. In some embodiments, the system can include a plurality of containers, a detecting agent in each of the plurality of containers, and an antimicrobial agent in at least a portion of the plurality of containers. The antimicrobial agent can be disposed in at least one of the plurality of containers. Each of the containers can contain at least a portion of the biological sample. The detecting agent can be configured to produce optically detectable changes responsive to bacterial respiration or growth.

In any of the embodiments disclosed herein, a first concentration of the antimicrobial agent and a first portion of the biological sample can be disposed in a first container of the plurality of containers, and a second concentration of the antimicrobial agent and a second portion of the biological sample can be disposed in a second container of the plurality of containers. The first concentration of the antimicrobial agent can reduce bacterial respiration or growth in the first portion of the biological sample by a first amount, and the second concentration of the antimicrobial agent can reduce bacterial respiration or growth in the second portion of the biological sample by a second amount.

In any of the embodiments disclosed herein, a first portion of the detecting agent can be disposed in the first container and can produce a first optically detectable change in the first container, and a second portion of the detecting agent can be disposed in the second container and can produce a second optically detectable change in the second container.

In any of the embodiments disclosed herein, the system can include an imaging device configured to detect the optically detectable change. The optically detectable change can include changes in color or turbidity or both.

In any of the embodiments disclosed herein, the detecting agent can include an oxygen-sensitive chemical group.

In any of the embodiments disclosed herein, the detecting agent can include a chromophore in solution.

In any of the embodiments disclosed herein, the detecting agent can include a chromophore encapsulated within a carrier of porous hydrogel, silica, microparticles, or nanoparticles.

In any of the embodiments disclosed herein, the detecting agent can include a chromophore immobilized on the surface of a carrier of porous hydrogel, silica, microparticles, or nanoparticles.

In any of the embodiments disclosed herein, the detecting agent can include one or more of: oxyhemoglobin, hemoglobin, myoglobin, leuco-dyes, allotropes of carbon, metalloporphyrins, and oxygen sensing dyes.

In any of the embodiments disclosed herein, the system can further include an incubator configured to incubate the bacteria.

Another exemplary embodiment of the present disclosure provides a method of detecting antimicrobial resistance in a biological sample from a subject. The method can include combining a first portion of the biological sample with a first concentration of an antimicrobial agent in a first container, combining a second portion of the biological sample with a second concentration of the antimicrobial agent in a second container, measuring a first optical property from the first container, and measuring a second optical property from the second container.

In any of the embodiments disclosed herein, the method can further include mixing a detecting agent with the sample from a subject.

In any of the embodiments disclosed herein, the detecting agent can include one or more of: oxyhemoglobin, hemoglobin, myoglobin, leuco-dyes, allotropes of carbon, metalloporphyrins, and oxygen sensing dyes.

In any of the embodiments disclosed herein, measuring the first optical property and the second optical property can include capturing an image of the first container and an image of the second container respectively after a passing of a time interval following combining.

In any of the embodiments disclosed herein, the method can further include comparing the first optical property and the second optical property to a control optical property from a control well in which there can be a third portion of biological sample and determining an inhibition of bacterial growth or presence in the biological sample based on the comparing.

Another exemplary embodiment of the present disclosure provides a method for determining a minimum inhibitory concentration of an antimicrobial agent. The method can include combining each of a plurality of portions of a biological sample with a plurality of respective varying concentrations of an antimicrobial agent and a detecting agent configured to produce optically detectable changes responsive to bacterial respiration, placing the plurality of portions in a plurality of respective containers, measuring an optical property of each container, and determining a minimum inhibitory concentration from the plurality of concentrations based on the optical property.

In any of the embodiments disclosed herein, determining the minimum inhibitory concentration can include comparing the optical property of each container to an optical property measured from a control container and, based on the comparing, determining at least one concentration at which bacterial growth is inhibited, wherein the minimum inhibitory concentration is the lowest concentration of the at least one concentration.

In any of the embodiments disclosed herein, measuring the optical property can include capturing an image of the plurality of containers periodically following combining.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides conventional susceptibility testing methods.

FIG. 2 provides a sample-antibiotics distribution scheme, in accordance with an exemplary embodiment of the present invention.

FIG. 3 provides an image of 96-well plates containing samples with progression of time, in accordance with an exemplary embodiment of the present invention.

FIG. 4A illustrates the change in color after incubation in growth media extracted from BacT/ALERT blood culture bottles for 4 hours, in accordance with an exemplary embodiment of the present invention.

FIG. 4B illustrates the change in turbidity after incubation in growth media extracted from BacT/ALERT blood culture bottles in standard BMD for 18 hours, in accordance with an exemplary embodiment of the present invention.

FIG. 5 illustrates the change in color of blood in growth media extracted from BacT/ALERT blood culture bottles before purging with CO₂, after purging with CO₂, and with purging with O₂, in accordance with an exemplary embodiment of the present invention.

FIG. 6A provides UV-Vis spectra of human whole-blood in growth media extracted from BacT/ALERT blood culture bottles before purging with CO₂ (bottom), after purging with CO₂ (middle), and with purging with O₂ (top), in accordance with an exemplary embodiment of the present invention.

FIG. 6B provides UV-Vis spectra of human whole-blood in CAMHB (cation-adjusted Mueller-Hinton broth) before purging with CO₂ (bottom), after purging with CO₂ (middle), and with purging with O₂ (top), in accordance with an exemplary embodiment of the present invention.

FIG. 7 provides an absorption spectrum of 2 to 20 μL of human whole-blood in media that was extracted from BacT/ALERT with the final volume of 200 μL for each sample, with inset showing near-IR absorption, and with purging with O₂, in accordance with an exemplary embodiment of the present invention.

FIG. 8A illustrates the change in color of bacteria-antibiotics combined in a 96-well plate for 0 hours, in accordance with an exemplary embodiment of the present invention.

FIG. 8B illustrates the change in color of bacteria-antibiotics combined in a 96-well plate after 24 hours, in accordance with an exemplary embodiment of the present invention.

FIG. 9 provides absorption spectra of wells in FIG. 8B after 24-hour incubation. were used, in accordance with an exemplary embodiment of the present invention.

FIG. 10 provides an image of 96-well plates immediately after distribution of sample, in accordance with an exemplary embodiment of the present invention.

FIG. 11A provides an image of 96-well plates showing the standard BMD from purified culture at 0 hours, in accordance with an exemplary embodiment of the present invention.

FIG. 11B provides an image of 96-well plates showing the standard BMD from purified culture at 24.5 hours, in accordance with an exemplary embodiment of the present invention.

FIG. 11C provides an image of 96-well plates showing colorimetric assays after distributing the 4.5 hour incubated spiked blood at 0 hours, in accordance with an exemplary embodiment of the present invention.

FIG. 11D provides an image of 96-well plates showing colorimetric assays after distributing the 4.5 hour incubated spiked blood at 24.5 hours, in accordance with an exemplary embodiment of the present invention.

FIG. 12 provides UV-Vis spectra ranging from 700 to 995 nm of each well corresponding to the colorimetric wells of FIG. 11D, in accordance with an exemplary embodiment of the present invention.

FIG. 13 provides a schematic diagram showing two-fold dilution of antibiotics with Rows A, B, and C showing distribution of ceftazidime, rows D, E, and F showing distribution of meropenem, and amount of ceftazidime and meropenem in columns G and H, respectively, in accordance with an exemplary embodiment of the present invention.

FIG. 14A provides an image of a 96-well plate showing standard BMD method at 18 hours, in accordance with an exemplary embodiment of the present invention.

FIG. 14B provides an image of a 96-well plate showing the minimum inhibitory concentration (MIC) assays directly from positive blood culture 18 hours, in accordance with an exemplary embodiment of the present invention.

FIG. 15 provides UV-Vis spectra ranging from 500 to 700 nm of each well corresponding to the colorimetric wells of FIG. 14B, in accordance with an exemplary embodiment of the present invention.

FIG. 16 provides a schematic of 96-well plate bacteria and antibiotics distribution (top) and a photo illustrating the layout of the actual microwell plate with blood/bacteria and antibiotic in each well at time 0 hours (bottom), in accordance with an exemplary embodiment of the present invention.

FIG. 17 provides images of 96-well plates showing MIC determination of Mu890 clinical isolate using blood as contrast reagent, in accordance with an exemplary embodiment of the present invention.

FIG. 18A provides a bar plot of different type of Gram-negative bacteria studied and their corresponding number, in accordance with an exemplary embodiment of the present invention.

FIG. 18B provides a boxplot showing bacterial concentration (CFU/mL) in the positive blood culture of each type of Gram-negative bacteria, in accordance with an exemplary embodiment of the present invention.

FIG. 19 provides acquired images at various times, in accordance with an exemplary embodiment of the present invention.

FIG. 20A provides a plot of a discriminant function plotted versus time, in accordance with an exemplary embodiment of the present invention.

FIG. 20B provides a set of images of color difference between positive and negative-control wells over time, in accordance with an exemplary embodiment of the present invention.

FIG. 21A provides a schematic showing color discriminate function triggering real-time analysis, in accordance with an exemplary embodiment of the present invention.

FIG. 21B provides Bayesian updated decision surface and output minimum inhibitory concentrations at various time points, in accordance with an exemplary embodiment of the present invention.

FIG. 22 provides shows Bayesian updated predicted labels by FAST SVM, in accordance with an exemplary embodiment of the present invention.

FIG. 23 provides a plot comparing error between an example assay in accordance with an exemplary embodiment of the present invention and standard BMD.

FIG. 24A provides a bar-plot showing average essential agreement (EA) for a variety of antibiotics with reference BMD, in accordance with an exemplary embodiment of the present invention.

FIG. 24B provides a bar-plot showing the average categorical agreement (CA) of a variety of antibiotics over 65 isolates with reference BMD, in accordance with an exemplary embodiment of the present invention.

FIG. 25A provides EA for the present assay with respect to BMD and corrected BMD, in accordance with an exemplary embodiment of the present invention.

FIG. 25B provides EA for the present assay with respect to BMD and corrected BMD, in accordance with an exemplary embodiment of the present invention.

FIG. 26 provides a flowchart for a method of detecting antimicrobial resistance in a biological sample from a subject, in accordance with an exemplary embodiment of the present invention.

FIG. 27 provides a flowchart for a method of determining a minimum inhibitory concentration of an antimicrobial agent, in accordance with an exemplary embodiment of the present invention.

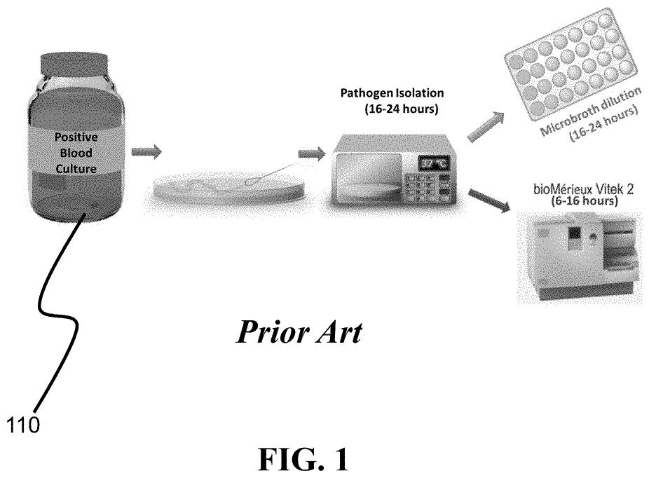

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Though the terms “bacteria”, “bacterium”, and “bacterial” are used herein, the present disclosure can also be applied to other microorganisms such as fungi and others.

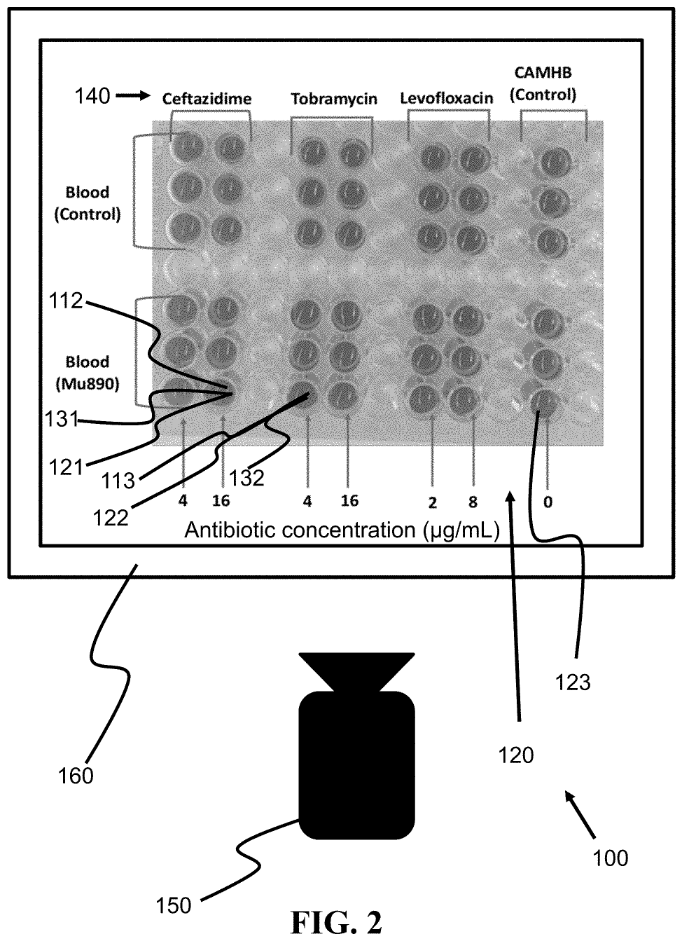

As shown in FIG. 2, an exemplary embodiment of the present disclosure provides a system 100 for detecting antimicrobial susceptibility of a bacteria 111 in a biological sample 110. In some embodiments, the system can include a plurality of containers 120, a detecting agent 130 in each of the plurality of containers 120, and an antimicrobial agent 140 in at least a portion of the plurality of containers 120. The antimicrobial agent 140 can be disposed in at least one of the plurality of containers 120. Each of the containers can contain at least a portion of the biological sample 110. The detecting agent 130 can be configured to produce optically detectable changes responsive to bacterial respiration or growth.

In any of the embodiments disclosed herein, a first concentration of the antimicrobial agent and a first portion 112 of the biological sample 110 can be disposed in a first container 121 of the plurality of containers 120, and a second concentration of the antimicrobial agent and a second portion 113 of the biological sample 110 can be disposed in a second container 122 of the plurality of containers 120. The first concentration of the antimicrobial agent 140 can reduce bacterial respiration in the first portion 112 of the biological sample 110 by a first amount, and the second concentration of the antimicrobial agent 140 can reduce bacterial respiration in the second portion 113 of the biological sample 110 by a second amount. In some embodiments, the first amount can be zero or undetectable, and the second amount can be non-zero and detectable such that the there is a binary distinction between the first and second amount, namely growth versus no growth.

In any of the embodiments disclosed herein, a first portion 131 of the detecting agent 130 can be disposed in the first container 121 and can produce a first optically detectable change in the first container 121, and a second portion 132 of the detecting agent 130 can be disposed in the second container 122 and can produce a second optically detectable change in the second container 122. The differences in the optically detectable changes between the various containers in the plurality of containers 120 can indicate the effect the antimicrobial agent 140 has on bacterial respiration and/or growth in the respective containers. Thus, by placing differing amounts of the antimicrobial agent 140 in the various containers and observing the optically detectable change, e.g., a color change, a user of the system 100 can determine the amount of the antimicrobial agent 140 needed to inhibit bacterial growth and/or respiration. This, in turn, can lead to more appropriate antimicrobial agent dosing in a patient with a bacterial infection in a manner that reduces the potential for AMR. While the term change is used herein, in some examples the first optically detectable change in the first container can be undetectable from the baseline such that there is a binary distinction between the optically detectable changes in the first container and the second container.

In any of the embodiments disclosed herein, the system 100 can include an imaging device 150 configured to detect the optically detectable change. The optically detectable change can include changes in color, turbidity, absorption, or extinction. Absorbance, for example, absorbance at from between about 200 nm through about 1000 nm can also be used. Single wavelengths, single wavelength ranges, multiple wavelengths, multiple wavelength ranges, or continuous spectra over any subset of this range can be used.

In any of the embodiments disclosed herein, the detecting agent 130 can include an oxygen-sensitive chemical group.

In any of the embodiments disclosed herein, the detecting agent 130 can include a chromophore in solution.

In any of the embodiments disclosed herein, the detecting agent 130 can include a chromophore encapsulated within a carrier of porous hydrogel, silica, microparticles, or nanoparticles.

In any of the embodiments disclosed herein, the detecting agent 130 can include a chromophore immobilized on the surface of a carrier of porous hydrogel, silica, microparticles, or nanoparticles.

In any of the embodiments disclosed herein, the detecting agent 130 can include one or more of: oxyhemoglobin, hemoglobin, myoglobin, leuco-dyes, allotropes of carbon, metalloporphyrins, and oxygen sensing dyes. In any of the embodiments disclosed herein, the detecting agent 130 can include Lumbricus terrestris hemoglobin, polymerized or cross-linked hemoglobin. Lumbricus terrestris is also known as the earthworm. Other worm hemoglobin can also be used, as could those from other animals, such as horses. Earthworm hemoglobin is much larger and more stable when compared to human hemoglobin. These detecting agents can be isolated from any animal or human. Whole blood can also be used as the detecting agent 130. In any of the embodiments disclosed herein, the system can further include an incubator 160 configured to incubate the bacteria 111.

As shown in FIG. 33, an exemplary embodiment of the present invention provides a method 200 of detecting antimicrobial resistance in a biological sample from a subject. The method can include at step 202 combining a first portion of the biological sample with a first concentration of an antimicrobial agent in a first container, at step 204 combining a second portion of the biological sample with a second concentration of the antimicrobial agent in a second container, at step 206 measuring a first optical property from the first container, and at step 208 measuring a second optical property from the second container.

In any of the embodiments disclosed herein, the method can further include mixing a detecting agent with the sample from a subject.

In any of the embodiments disclosed herein, the detecting agent can include one or more of: oxyhemoglobin, hemoglobin, myoglobin, leuco-dyes, allotropes of carbon, metalloporphyrins, and oxygen sensing dyes.

In any of the embodiments disclosed herein, measuring the first optical property and the second optical property can include capturing an image of the first container and an image of the second container respectively after a passing of a time interval following combining.

In any of the embodiments disclosed herein, the method can further include comparing the first optical property and the second optical property to a control optical property from a control well in which there can be a third portion of biological sample and determining an inhibition of a bacterial growth or presence in the biological sample based on the comparing. In some examples, the optical property can be turbidity or scattered light using spectral and/or RGB values from a color charge-coupled device (CCD) camera used for imaging.

As shown in FIG. 34, an exemplary embodiment of the present invention provides a method 300 for determining a minimum inhibitory concentration of an antimicrobial agent. The method can include at step 302 combining each of a plurality of portions of a biological sample with a plurality of respective varying concentrations of an antimicrobial agent and a detecting agent configured to produce optically detectable changes responsive to bacterial respiration, at step 304 placing the plurality of portions in a plurality of respective containers, at step 306 measuring an optical property of each container (with or without a detecting agent added), and at step 308 determining a minimum inhibitory concentration from the plurality of concentrations based on the optical property.

In any of the embodiments disclosed herein, determining the minimum inhibitory concentration can include comparing the optical property of each container to an optical property measured from a control container and, based on the comparing, determining at least one concentration at which bacterial growth is inhibited, wherein the minimum inhibitory concentration is the lowest concentration of the at least one concentration.

In any of the embodiments disclosed herein, measuring the optical property can include capturing an image of the plurality of containers periodically following combining.

The following examples further illustrate aspects of the present disclosure. However, they are in no way a limitation of the teachings or disclosure of the present disclosure as set forth herein.

### Example 1

In some embodiments, the tests described herein demonstrates a fast, simple, and easy to use test that can outperform tests on the market. The tests can also provide a low labor cost. A rapid, spectroscopic or colorimetric, phenotypic antibiotic susceptibility test (AST) for bloodstream infections (BSIs) and other bodily fluid infections has been developed that can enable initiation of appropriate treatment within 3-24 hours after initial blood draw. Phenotypic ASTs are preferred over genetic tests as typically only phenotypic methods can detect novel and emerging resistance of bacteria towards antibiotics.

The approach described herein can apply to urine, sputum, cerebrospinal fluid, blood, blood culture, and all raw and cultured bodily fluids/specimens. BSIs are typically the most time consuming and difficult to analyze as bacteremia and sepsis patients typically have very low bacterial content of ≤100 colony forming units per milliliter (CFU/mL), buried within ˜3×10⁹ red blood cells/mL. Blood culture is typically needed to enrich bacteria to detectable levels of 10⁷-10⁹ CFU/mL. The inventors' AST is direct from positive blood culture and eliminates the plating/isolation step needed for standard ASTs. This approach relies on diluting positive blood culture into CAMHB media with antimicrobials at varying concentrations. Microbial growth is determined spectrally or colorimetrically using either turbidity or added contrast agents that are sensitive to bacterial respiration and growth. The inventors have combined the AST and culturing steps with a respiration-sensitive or oxygen-sensitive dye to decrease time to result for ASTs with both spectroscopic or colorimetric readouts to provide an AST within 3-24 hours from initial blood draw. This rapid AST can shave many hours (even days) off the time currently needed for BSIs, and can be compatible with clinical workflows. The automated colorimetric readout in a microwell plate is suitable for use in both high and low resource environments.

A high throughput colorimetric assay with minimal sample preparation and handling will minimize the susceptibility timelines, directly improve patient outcomes, and suppress the alarming rate of antibiotic resistance infections.

As an initial demonstration that blood can be used as a colorimetric indicator of bacterial growth under antimicrobial pressure, whole blood was spiked with blood-stable bacteria and used in this study to mimic the low bacterial CFU/mL. As a control, 100 μL of pure blood was plated on LB agar, followed by incubation at 37° C. for 15 hours to ensure no bacteria were present prior to spiking. The CLSI sensitive and resistance breakpoints were used for testing the susceptibility of bacteria towards the selected antibiotics. At the initial stage, clinical isolates of E. coli strain Mu890 and its susceptibility with the ceftazidime, meropenem, and levofloxacin antibiotics was studied. Mu890 is an exemplary E. coli bacterial strain.

A single colony of Mu890 was picked and used as the inoculum for growth in 4 mL pre-autoclaved Cation-Adjusted Mueller-Hinton Broth (CAMHB) media for 3 hours. After 3 hours, the optical density at 600 nm (OD-600) was measured, and the sample was diluted to obtain 200 CFU/mL bacteria density. 1 mL of 200 CFU/mL of bacteria was added to the 1 mL of whole blood to obtain initial bacterial content of about 100 CFU/mL while the control contains 1 mL of whole blood and 1 mL of CAMHB media. This mixture was equilibrated for 30 minutes by shaking at 225 rpm at 37° C., followed by plating 100 μL of equilibrated sample in the LB agar plate which was incubated for 14 hours. After equilibration, 1 mL of each sample was mixed with 3 mL of BacT/ALERT blood culture medium followed by incubation for 7 hours at 225 rpm and 37° C. and plating the 100 μL of diluted simulated blood culture after incubation. The Mu890 density after 30 minutes equilibration and 4.5 hours incubation are shown in Table 1.

TABLE 1
Evaluation of Mu890 density after 30 minutes
equilibration and 7 hours incubation.

| | Blood Culture with | Blood control |
|---|---|---|
| Sample | Mu900 (CFU/mL) | (CFU/mL) |
| After 30 minutes equilibration | 80 | 0 |
| After 7 hours incubation | 12 × 10⁴ | 0 |
| Bacteria amount in each well | 6 × 10⁴ | 0 |

After 4.5-hr incubation to generate enough bacteria to split among wells, 100 μL distributed into each well of a 96-well plate, which had been pre-filled with the appropriate antibiotics solution to achieve the final antibiotics concentration indicated. Microwell plates were covered with a sterile film to seal the wells. The antibiotics distribution scheme is shown in FIG. 2.

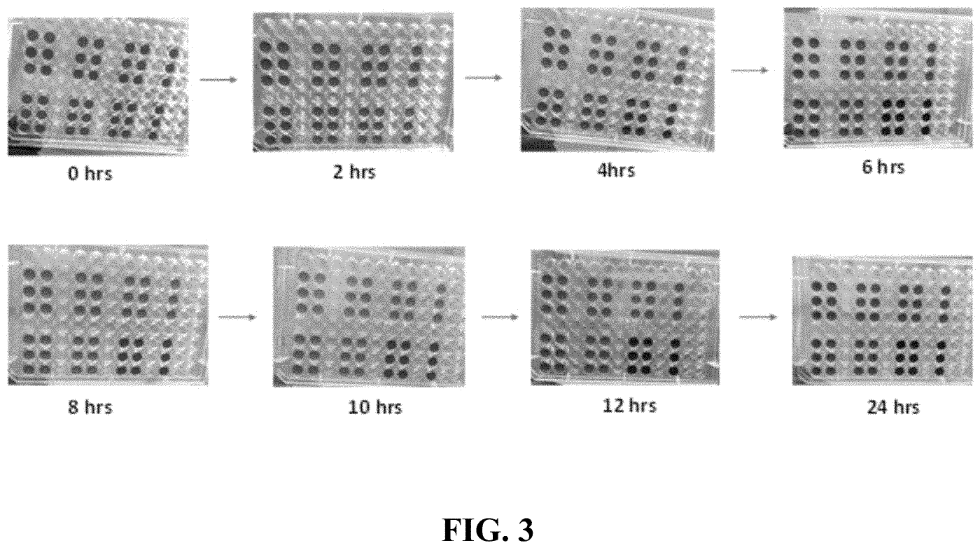

After distribution of Mu890 in the 96-well plate, the plate was incubated at 37° C., with shaking at 225 rpm. The color of the incubated sample was monitored by taking photos with the progress of time, shown in FIG. 3. FIG. 3 is the bottom view of the plate.

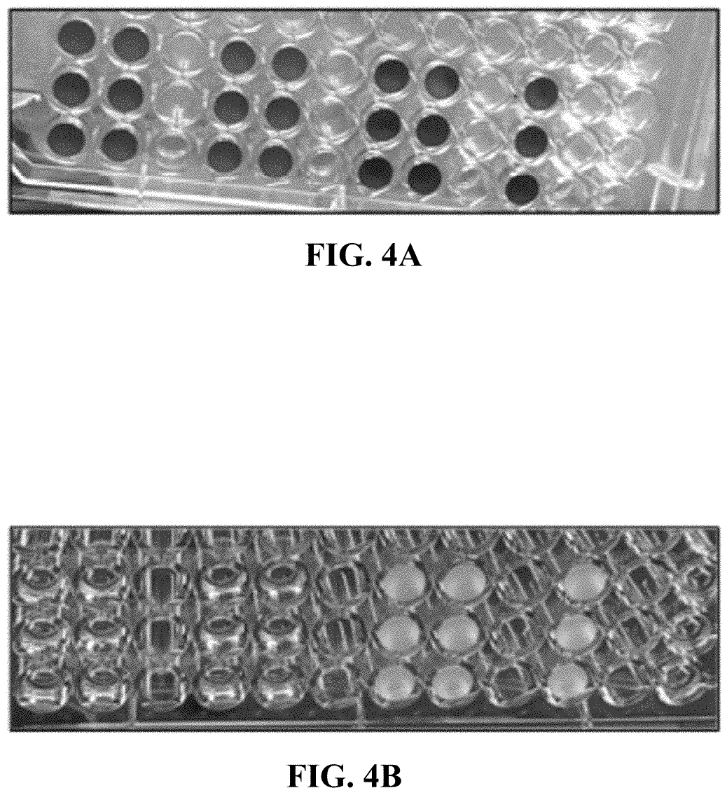

As evident from FIG. 3, the wells containing Mu890 and CAMHB showed change in color from bright red to dark red. This color change results from the growth and presence of viable bacteria. When bacteria are resistant to the given antibiotic concentration, the color change is clearly observed. According to CLSI guideline, ceftazidime, tobramycin, and levofloxacin can be used for treatment of E. coli provided the bacteria is susceptible to the concentration presented in the guideline. The color change can be seen within ˜4 hours of incubation of plates indicating that E. coli strain, Mu890, grew and was resistant to levofloxacin. Wells containing ceftazidime and tobramycin do not show significant color change even after 24 hours incubation indicating that Mu890 is sensitive to both antibiotics. Results were validated by comparing with the gold standard BMD (FIGS. 4A and 4B). The gold standard BDM requires 16 to 24 hours incubation of plates after isolation of pure bacteria from positive blood cultures. In this case, the BMD was done by subculturing a pure colony of Mu890 in 4 mL CAMHB for 3 hours, followed by OD-600 adjustment such that the final concentration of bacteria is 6×10⁶ CFU/mL per well. The plate was incubated at 37° C. for 18 hours and the image was acquired (FIG. 4B). Both the rapid colorimetric AST and the BMD disclosed herein yield the same results, indicating Mu890 resistance towards levofloxacin and susceptibility to ceftazidime and tobramycin (FIGS. 4A and 4B).

FIG. 3 shows the color change with time due to bacterial growth when resistant to antibiotics. The color change results from conversion of oxyhemoglobin to deoxyhemoglobin due to bacterial respiration consuming O₂ and producing CO₂. Thus, this is a label-free, colorimetric indicator that is naturally present in blood that allows susceptibility to be determined under the present AST conditions. The oxyhemoglobin to deoxyhemoglobin change results in a spectral shift and can be used as an indicator in blood, where it is an endogenous chromophore in blood culture, and can be added to urine and other bodily fluid/specimen cultures. The top of the multi-well plates should be sealed with film to limit oxygen exchange with the environment. Thus, using oxygen-sensitive chromophores including whole blood, hemoglobin, myoglobin, leuco-dyes or other oxygen sensing colorimetric indicators, including triplet states and their temporal decays such as C₆₀, C₇₀ and other allotropes of carbon, metalloporphyrins, and dyes like methylene blue or rose bengal that exhibit large triplet quantum yields could all be suitable colorimetric or spectroscopic indicators for bacterial growth in the presence of antibiotics. Such dyes could be used free in solution or packaged within and/or on the surface of porous hydrogel, silica, or other nano- or micro-particles or surfaces. These contrast agents sensing products of bacterial growth could be added to weakly or non-colored samples to assess bacterial growth and therefore susceptibility in a wide variety of cultured or raw specimens or fluids.

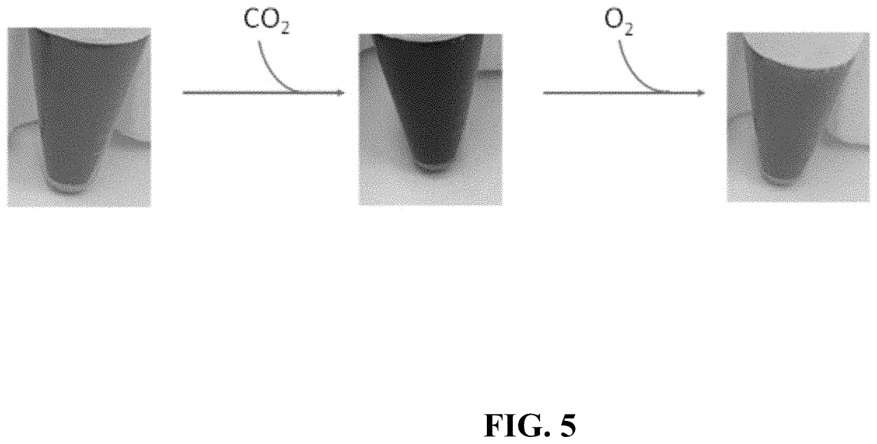

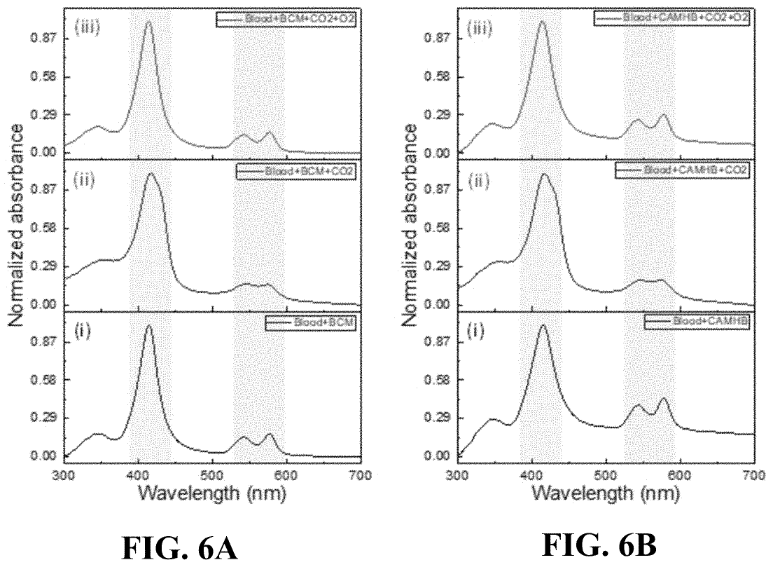

Multiple studies have been performed for diagnosis of bacterial presence in blood by analyzing the production of CO₂ in blood culture bottles. However, the susceptibility determination using the oxy-to-deoxyhemoglobin colorimetric approach has not been previously demonstrated. As shown in FIG. 5, purging of blood diluted in media extracted from BacT/ALERT blood culture bottles with CO₂ leads to color change, which on additional purging with O₂ reverses the color to that of the original blood culture. This color change was further investigated using UV-Vis and the corresponding spectra are shown in FIG. 6.

UV-Vis absorption spectra (FIG. 6) were taken by mixing 5 μL of blood culture in 1 mL phosphate-buffered saline (PBS). Several representative spectra are shown in FIG. 6 to show the spectral differences giving the observed color change. FIG. 6A(i) and B(i) show the sharp Soret band peaks of hemoglobin (Hb) at 414 nm which broadens on purging with CO₂ with appearance of new shoulder at 430 nm (FIG. 6A(ii) and 6B(ii)), however, on re-purging with O₂ the Soret band peaks show reappearance of sharp absorption at 414 nm. Similarly, the β and α bands of oxygenated Hb at 543 and 577 nm, respectively (highlighted by green band, FIG. 6), merge on deoxygenation and reappear on oxygenation. Despite having similar spectral change with bacterial growth as with CAMHB only, BacT/ALERT culture media was used for the colorimetric assay as it promotes faster growth and the color change.

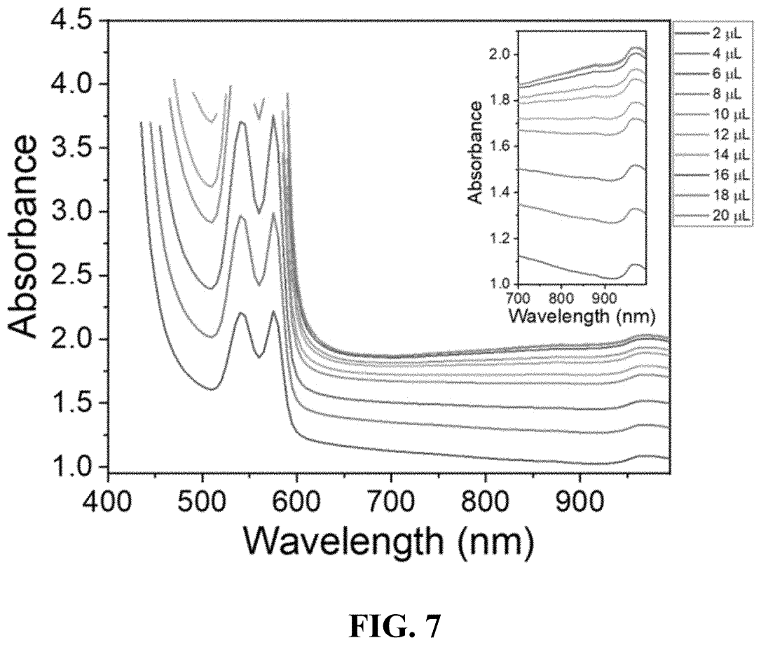

Color (visual) changes or spectral changes in the visible region have been demonstrated to rapidly indicate susceptibility or resistance through bacterial growth-induced changes in the oxy-/deoxy-hemoglobin equilibrium. This gives a straightforward colorimetric assay for a label-free AST, amenable even to low resource environments. Spectral changes, however, are likely to yield faster results, especially when coupled with machine learning or software-based analyses. The visible absorption can be too strong in the raw AST to allow this at the blood concentrations used, but absorption in the near IR also provides contrast between oxy- and deoxy-hemoglobin, with greatly increased light transmission (FIG. 7). Thus, near infrared probing of absorption in multi-well plates offers an additional sensitive spectroscopic approach to perform the AST.

Monitoring near IR absorbance for developing Rapid AST: Near IR absorption spectra (range 700 to 995 nm) of the sample being analyzed does not saturate as shown in inset of FIG. 7. To investigate the near IR possibilities, screening experiments were conducted. Table 2 shows the combination of clinical isolates of bacteria and antibiotics used for the analysis.

TABLE 2
Bacteria and antibiotics combination used
for validation of near IR absorption.

| Clinical isolates | Antibiotics evaluated |
|---|---|
| E. coli (Mu890) | ceftazidime, tobramycin, meropenem, |
| | levofloxacin |
| P. aeruginosa (PA46) | cefazolin |

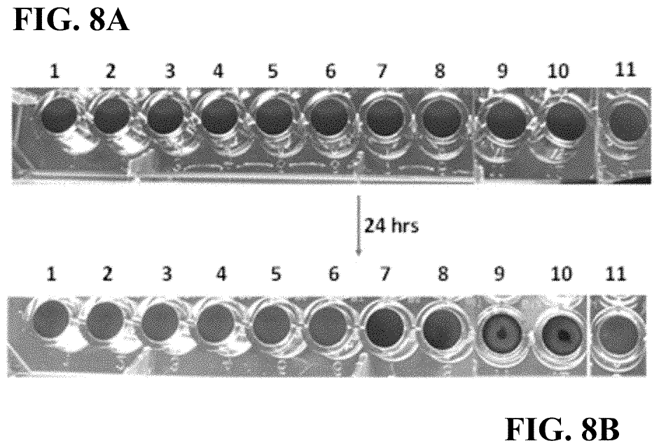

The experiment was conducted in a similar fashion to that described for the colorimetric assay, and spectra were taken from 700 to 990 nm after the samples were incubated at 37° C. for 24 hours in 96-well plate. The pictures before and after incubation are shown in FIGS. 8A and 8b. The colorimetric assays for bacteria shown in FIG. 8 were done in triplicate which yielded identical results; however, only single wells are shown here for convenience.

The antibiotics concentration and the initial concentration of bacterial cells in the wells shown in FIG. 8A are tabulated in Table 3. The darkening of original reddish color in FIG. 8B wells 7, 8, 9, and 10 indicate bacterial growth and that the bacteria are resistant towards the antibiotic concentrations used in those wells. On the other hand, the retention of color in FIG. 8B wells 1, 2, 3, 4, 5, 6, and 11 suggest that the bacteria are susceptible to the concentration of antibiotics used in those wells. Furthermore, these results were validated by comparing with the standard BMD method which gives the similar susceptibility breakpoints (picture not shown). According to both methods Mu890 is susceptible to ceftazidime, meropenem, tobramycin and is resistant to levofloxacin. Similarly, P. aeruginosa strain PA46 is resistant towards cefazolin.

TABLE 3
Clinical isolates, antibiotics and their concentration
in the corresponding wells shown in FIG. 8A and 8B.

| Clinical | CFU/mL in | | Well | CLSI breakpoints (μg/mL) | |
|---|---|---|---|---|---|
| isolates | the wells in A | Antibiotics | number | Susceptible | Resistant |
| Mu890 | 2.55 × 10⁴ | Ceftazidime | 1 | ≤4 | |
| | | | 2 | | ≥16 |
| | | Meropenem | 3 | ≤2 | |
| | | | 4 | | ≥4 |
| | | Tobramycin | 5 | ≤4 | |
| | | | 6 | | ≥16 |
| | | Levofloxacin | 7 | ≤2 | |
| | | | 8 | | ≥8 |
| PA46 | 1.85 × 10³ | Cefazolin | 9 | NA (Used 16) | |
| | | | 10 | | NA (Used 32) |
| Control | 0 | — | 11 | — | — |

The bacterial density in the standard BMD method was 6×10⁶ CFU/mL per well before incubation, while the bacterial density in the colorimetric assay before the incubation is given in Table 3. Furthermore, CLSI susceptible and resistant breakpoints of cefazolin which is used in the treatment of E. coli was also used to see its effect on simulated PA46 infected blood.

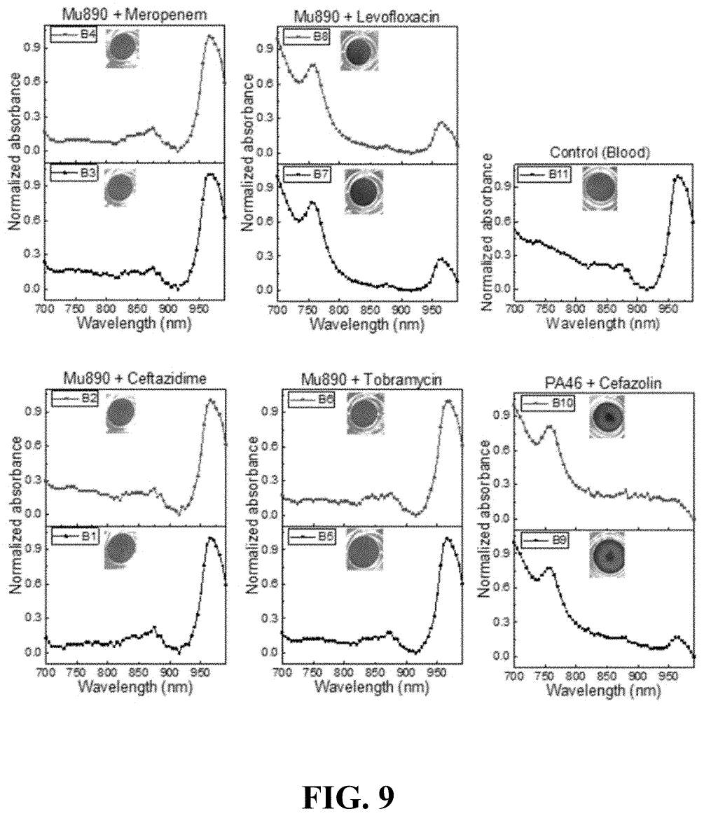

The absorbance spectra FIG. 9, for the colorimetric assay were taken after 24-hour incubation at 37° C. at 225 rpm (spectra of wells in FIG. 8B). FIG. 9 shows that the absorbance spectrum of bacteria susceptibility towards the antibiotics yields a decreased deoxygenated Hb (oxyHb) absorption at 755 nm as seen in B1, B2, B3, B4, B5, B6, and B11 wells. However, if the bacteria are resistant to the antibiotic in the well, the color changes leading to the appearance of the deoxygenated Hb (deoxyHb) band at 755 nm as seen in B7, B8, B9, and B10 wells in FIG. 9. The spectral data in FIG. 9 matches with the corresponding colorimetric assay. Hence, monitoring of these signal changes at 755 nm (or similar wavelengths) with the progress of time can improve sensitivity and reduce time for rapid AST from whole blood. Additionally, these spectral changes may give the AST result within few hours of plate incubation prior to observing pronounced color change. FIG. 9 Absorption spectra of wells in FIG. 8B after 24-hour incubation. Insets to each panel show color images of the corresponding wells from FIG. 8B. The 1st column gives absorption spectra of PA46. The 2nd-5th columns provide spectra of Mu890, and the 6th column gives the absorption spectrum of the control. The top half of each panel (red spectral curve) is the spectrum of bacteria in blood culture when exposed to the respective CLSI sensitive antibiotic concentrations. The black spectral curve (lower half of each panel) for each of the bacterial samples is when exposed to the respective CLSI resistant breakpoint. Spectral changes between red and black curves indicate a change in growth due to antibiotic concentration change. CLSI concentrations are shown in Table 3 and this experiment is in accordance with an exemplary embodiment of the present invention.

The final amount of blood in the 96-well plates will be crucial in designing the colorimetric AST from the infected human whole-blood. Thus, lower blood volume in the wells (<20 μL) will be appropriate for the colorimetric assay. Thus, this colorimetric or spectroscopic, high throughput, rapid AST is compatible with existing blood culture and AST procedures, but can simply provide the required information much faster, without affecting current practices. Thus, each rapid AST can be confirmed by standard (much slower) clinical methods, providing additional confirmation of results, while enabling more appropriate treatment to be administered at a much earlier stage.

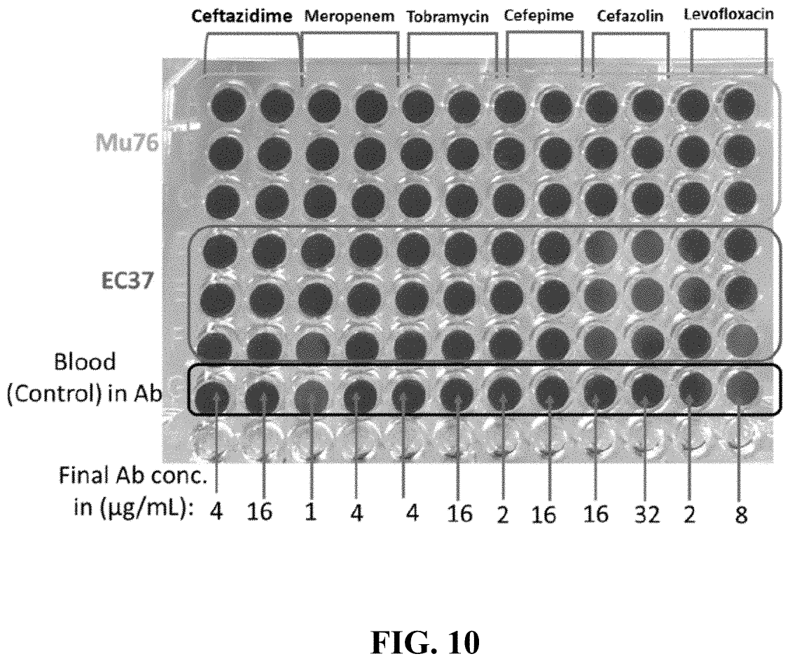

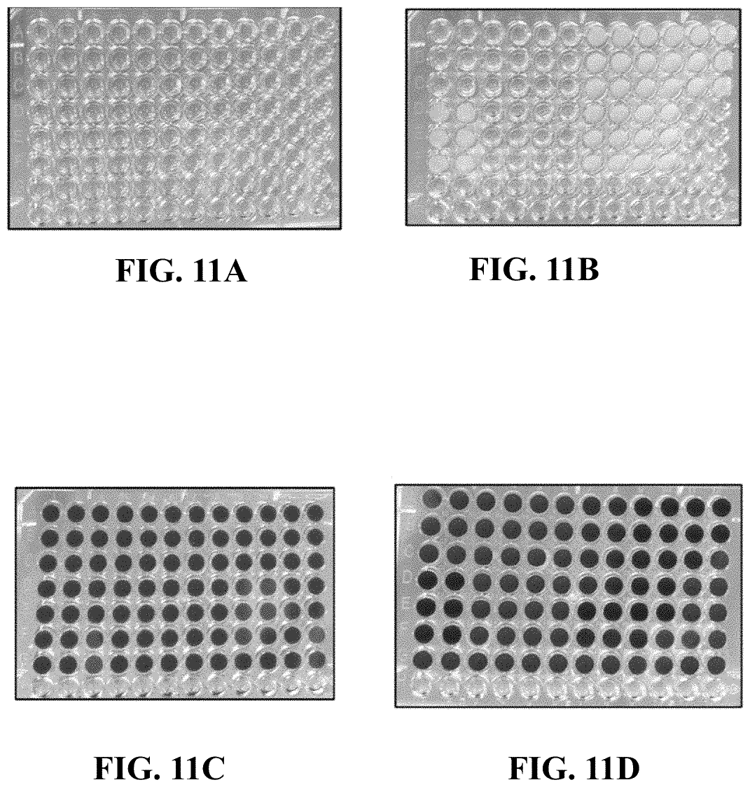

ASTs were also performed with clinical isolates Mu76 and EC37 starting with bacterial densities of ˜100 CFU/mL, spiked into 1 mL of human whole blood. This spiked blood was mixed 1:1 with 1 mL of CAMHB media and equilibrated for 30 minutes at 37° C. 1 mL of the equilibrated sample was then mixed with 3 mL growth medium followed by incubation for 4.5 h at 37° C., and 100 μL of the sample was distributed in a 96-well plate pre-filled with antibiotics. The photograph of the 96-well plate along with its contents is shown in FIG. 10. The final susceptible and resistant antibiotic concentration in the wells is in accordance with the CLSI susceptible and resistant breakpoints. After sample distribution, the plate was incubated at 37° C. The colorimetric assay was compared against the standard method (shown in FIG. 11). From FIGS. 11A-11D, it is evident that the colorimetric AST assays starting with 100 CFU/mL is in complete agreement with standard BMD method.

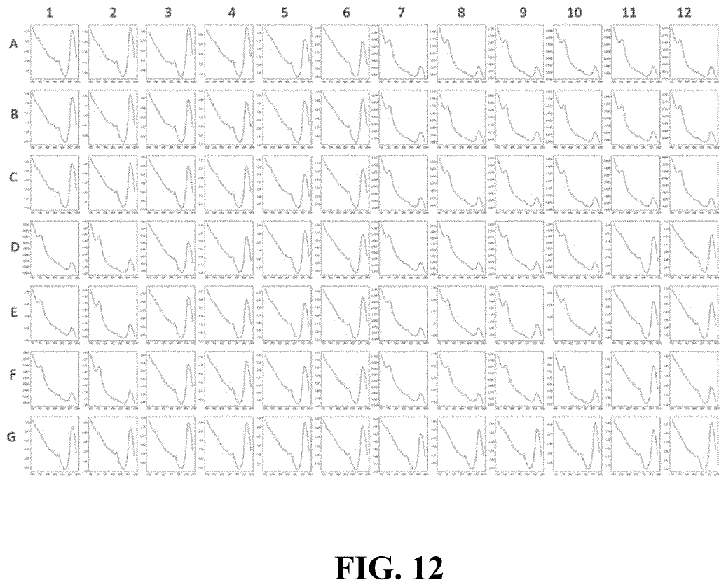

This assay can be performed as either a spectroscopic or colorimetric assay. FIG. 12 shows the representative spectra of FIG. 11D wells, with the spectral features being different in the visible (not shown here) and the near IR region (FIG. 12) for samples exhibiting bacterial growth, thereby performing the AST or MIC (minimum inhibitory concentration) determination.

FIG. 12 shows that in the wells where bacteria are resistant, the deoxyhemoglobin peak at 755 nm is pronounced whereas when the bacteria are susceptible there is no appearance of deoxyhemoglobin peak. This demonstrates that the inventors can not only determine MICs directly from drawn blood both spectroscopically and colorimetrically, but can also use blood or blood products as a colorimetric indicator of bacterial growth under antibiotic challenge.

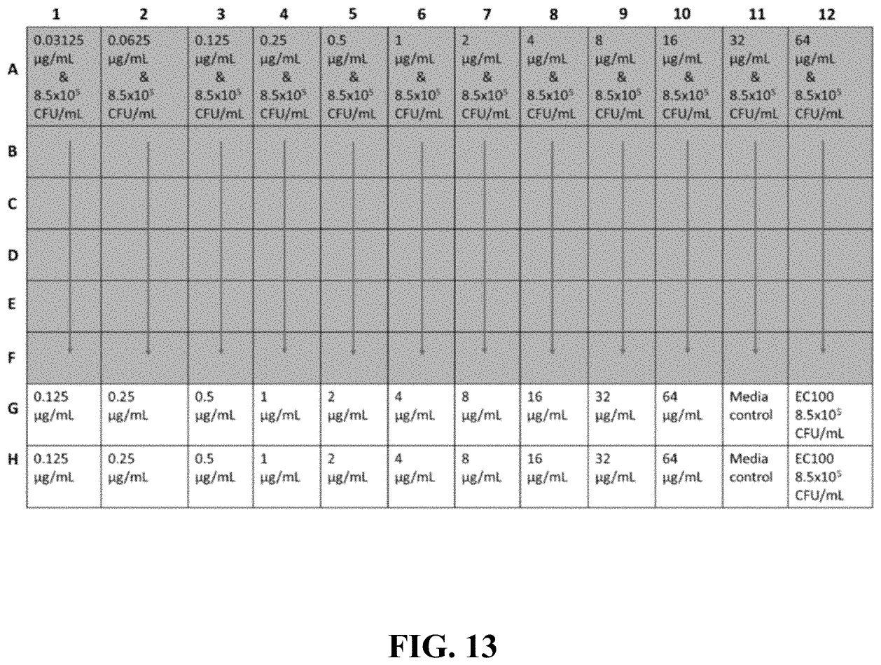

The standard method for MIC determination requires isolation of bacteria post-positive blood culture. The isolation of bacteria typically takes about 16 to 24 hours followed by MIC determination which typically requires an additional 16 to 24 hours for standard BMD and additional 6 to 16 hours for Vitek2. To remove the lengthy bacterial isolation and culture steps, the inventors performed MIC determinations directly from positive blood culture. The positive blood culture of an E. coli (EC100) isolate was used. The positive blood culture was diluted 200 times in CAMHB media which was then distributed in 96-well plates. The bacterial amount in the diluted sample was estimated by platting which was obtained to be 1.7×10⁶ CFU/mL. 100 μL of the diluted sample was distributed in the 96-well plate prefilled with antibiotics such that the bacterial amount on the well was approximately 8.5×10⁵ CFU/mL. The schematic diagram showing the two-fold antibiotics dilution, and the controls is shown in FIG. 13, where rows A, B, and C show the distribution of ceftazidime, and rows D, E, and F show the distribution of meropenem. Wells G1 to G10 shows ceftazidime and H1 to H10 shows meropenem controls without bacteria. Wells G11 and H11 contain media-only control. Wells G12 and H12 contain bacteria-only control.

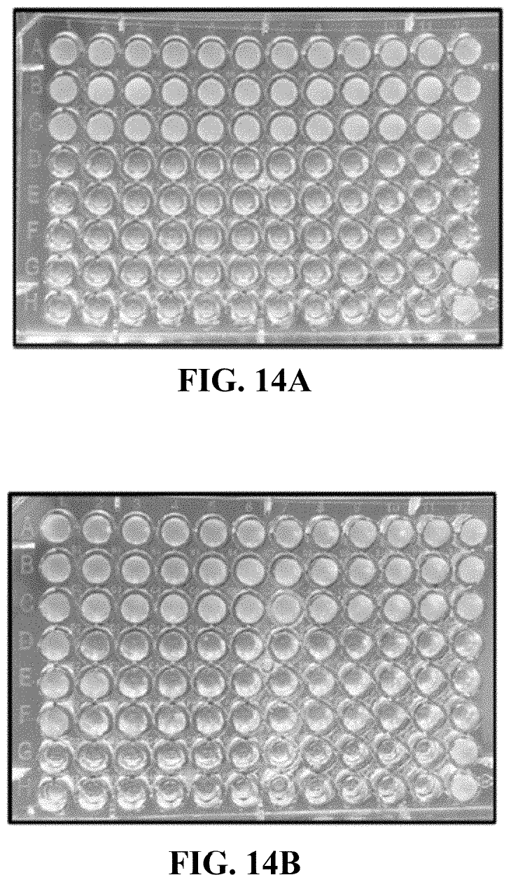

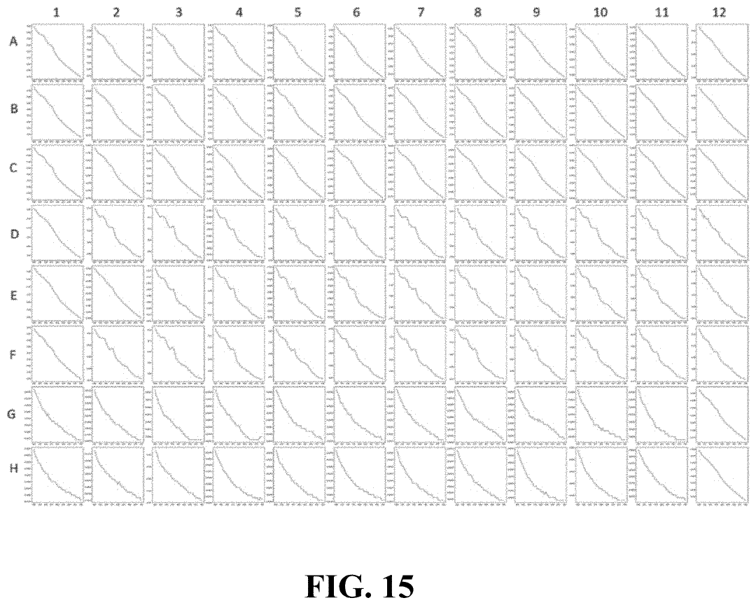

The MIC obtained directly from positive blood culture were compared with standard BMD (broth microdilution) which shows high agreement of 95% (only wells D1, E1, F1 and E2 did not match). The inoculum sizes for the two assays, however, are likely quite different. Adjusting these to be the same is likely to give even higher agreement as in example 2 below. Note that additional contrast agents such as those chromophores, dyes or nanoparticles listed above could be used, as could blood or blood products to give a colorimetric or spectroscopic endpoint or turbidity to give a scattered light signature throughout the visible and near infrared spectrum. In this case, the positive blood culture was diluted such that color is difficult to visualize. Thus, spectroscopic analyses can still be used, as can turbidity due to the high bacterial densities within 200-fold diluted positive blood cultures. The pictures for MIC determination directly from positive blood culture and standard BMD are shown in FIGS. 14A and 14B, showing that turbidity can also be used for determining MICs without prior isolation and purification, also saving precious time for MIC or AST determinations from positive cultures. The UV-vis absorption spectra for each of the wells in FIG. 14B are shown in FIG. 15, showing that weak hemoglobin bands at ˜543 nm and ˜577 nm can still be monitored in place of or in addition to turbidity for determining bacterial growth in the presence of antibiotics.

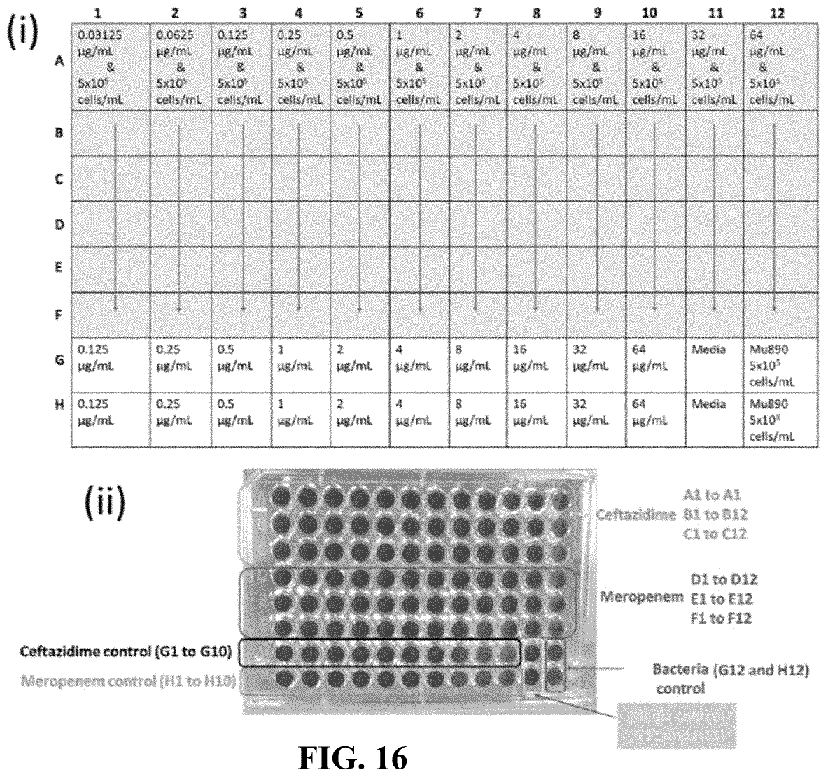

Blood or blood products as contrast reagent. To demonstrate that blood can be used as contrast agent for colorimetric assays to determine MIC, an experiment was conducted using the clinical isolate Mu890. Here, first the blood contrast reagent was prepared by mixing human blood in CAMHB media (10% v/v). 100 μL of thus prepared contrast reagent was distributed in a 96-well plate followed by serial dilution of antibiotics. Mu890 isolate was grown in CAMHB media, and the bacterial cell density were estimated by measuring the OD at 600 nm and the bacterial amount were adjusted such that the final bacterial amount per well is approximately 5×10⁵ cells/mL. The schematic of the 96-well plate layout is shown in FIG. 16.

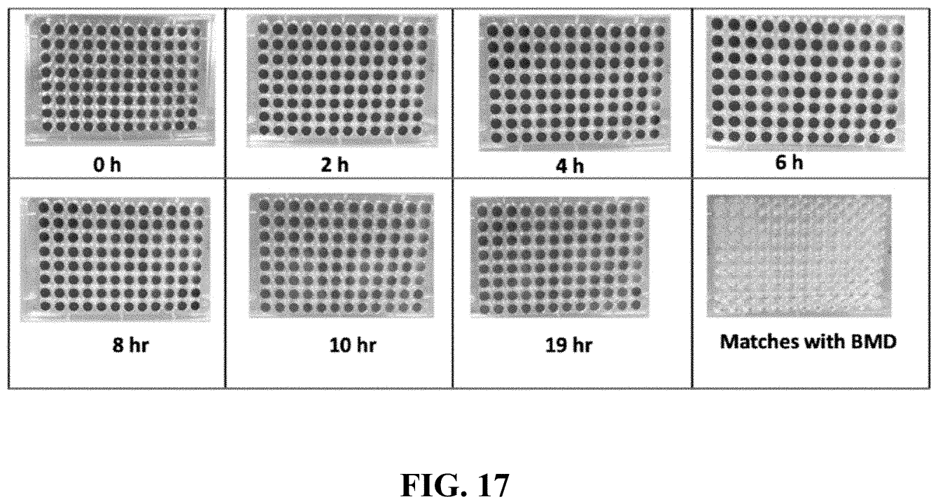

After distribution, the 96-well plate was incubated at 37° C. The MICs can be obtained within 4 h, as shown in FIG. 17. Thus, one should be able to determine MICs directly from infected blood after a ˜5-hour preincubation of initially <100 CFU/mL spiked blood in 1 mL total blood volume to determine the MIC within ˜9 hours of simulated blood draw. Lower initial CFU/mL in blood would require slightly longer preincubation steps, but MICs should be obtained in <12 hours from initial blood draw (vs. ˜60 hours for current methods).

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

In some embodiments, the rapid diagnostic test can use less than 3 mL of blood and provide a full antibiotic susceptibility test (AST) in as little as 7 hours from initial blood draw. Normally, antibiotic susceptibility testing takes around 60 hours from initial blood draw and is the limiting timescale for actionable treatment information. In the system and method described herein, a patient's blood can be drawn and injected into a standard blood culture medium. This infected blood can be incubated for about 4 hours to generate sufficient bacterial population, then dispersed into antibiotic-containing 96 or 384-well plates, and a film placed over the microwell plate. Bacterial growth induces a color change in the blood resulting from bacterial respiration only in the wells in which bacteria are resistant to the antibiotic concentration. A color change can be registered either by taking a photograph with a digital camera, visual inspection, or measuring the absorption changes due to bacterial growth in either the visible or in the near infrared spectral regions. This rapid antibiotic susceptibility test (AST) is highly accurate and can be compatible with existing, much slower ASTs because it uses so little blood. Spectroscopic signatures of the infected blood in each well can be measured for a rapid and quantitative determination and response. In low resource environments, plates can be visually inspected at various time intervals for the detection of a color change. Both susceptibilities and quantitative minimum inhibitory concentrations can be obtained. The color change results from oxy to deoxy-hemoglobin, and hemoglobin is naturally present in blood, making this approach completely label free. When bacterial growth is inhibited, this conversion is not triggered and the well remains red. While this phenomenon can be visually detected, it can also be detected by comparison of peaks located in the infrared region. ASTs in urine, sputum, or other bodily fluids/samples can be performed by adding in hemoglobin as an indicator or using a different oxygen-sensitive dye.

In some embodiments, the system and method use a very short blood culture (˜6 hours) to generate sufficient bacteria to split among wells in a microwell plate. This is typically insufficiently long for current blood cultures to indicate bacterial presence. The user disperses blood into growth media with and without antibiotics for another ˜3 hours. If bacteria grow, hemoglobin changes color. This gives a total AST time to result of ˜10 hours from initial patient sample collection >6 times faster than current procedures. This allows proper antibiotic treatment to be administered much more rapidly, improving patient outcomes and decreasing antibiotic resistance. Colorimetric (photographs) readout and/or spectrophotometric (absorption spectroscopy) can be readily performed. This approach can be applied to blood, urine, sputum, other bodily fluids/samples for much faster ASTs with minimal technician labor. Other oxygen sensing dyes could be used to replace hemoglobin. This can be performed in raw blood samples, without long incubation and no purification/isolation of bacteria being necessary. ASTs can be performed directly, giving susceptibility profiles of infecting bacteria directly. The colorimetric output should be easily performed even in low resource environments—just an incubator, normal biological consumables, light source, and phone camera would be needed. No complicated analyses are needed for fast accurate results. Instrumentation and software development will better quantify susceptibilities and improve overall throughput in higher resource settings.

In some embodiments, when admitted to the hospital, patients have their blood drawn and urinalysis performed to see if bacteria are present which could be the cause of their malaise, and potentially lead to sepsis (very serious). There are a large number of samples that need to be processed by the clinical microbiology labs in hospitals as bacteremia and sepsis are one of the top 10 causes of hospital deaths, and antimicrobial resistance proliferation is increasing, making treatment even more difficult. Faster processing of samples and identification of appropriate treatment requires faster ASTs to be developed, as slow ASTs are currently the limiting step. The present approach is technically simple, easy to implement in a high-throughput manner, and up to 6 times faster than current methods. This identifies the appropriate treatment >50 hours sooner than existing methods, improving patient outcomes and decreasing proliferation of antimicrobial resistance.

In some embodiments, advantages of the present disclosure include a system and method that is faster (6-fold faster), simpler (less technician labor), compatible with high throughput screening, colorimetric (easy to visualize color change) or quantitative spectrophotometric assay for accurate characterization/quantification, compatible with existing methods as very little blood needs to be used, allowing existing (slow) methods to be used in parallel for further confirmation, and allowing for expansion to other fluids/samples.

### Example 2

Bloodstream infections (BSIs) are a major cause of mortality and morbidity throughout the globe, affecting 30 million people and causing 6 million deaths annually. BSIs directly cause sepsis—an acute immune response to the extremely low microorganism levels in infected blood that results in ˜350,000 deaths annually in the US alone. Unfortunately, BSIs are quite common, and the fraction caused by highly resistant bacteria that evade often unsuitable empiric treatments are especially dangerous. Broad resistance is particularly problematic in Gram negative bacteria as they account for >50% BSIs, but their susceptibility profiles are less-readily inferred from genetic or other rapid tests available. As time to appropriate treatment is the major determinant of patient survival, both the high BSI-related death toll and antimicrobial resistance proliferation could be significantly attenuated by rapid antimicrobial susceptibility tests (ASTs) that identify the most appropriate treatment at the earliest stages. Such rapid phenotypic susceptibility testing would also lower patient and overall healthcare costs, with sepsis treatment in the US alone accounting for total hospital costs of $24 billion annually.

Rapid BSI detection is challenged by the extremely low bacterial loads of about 1 to about 10 CFU/mL, masked by the very high numbers of blood cells of greater than about 10⁹/mL. Thus, approximately 24-hour blood culture is typically needed to amplify bacterial numbers to confirm infection, identify the pathogen, and determine susceptibility. After blood culture positivity, susceptibility is the slowest step, with clinically approved methods typically requiring plating taking approximately 18 hours, colony selection and resuspension, followed by growth-based susceptibility testing taking approximately 12 hours, resulting in a minimum delay of approximately 30 hours after blood culture has turned positive before appropriate treatment is determined. These current clinical implementations are faster than broth microdilution (BMD) or disk diffusion, the gold standard phenotypic susceptibility tests recommended by the CLSI, both of which require approximately 48 hours from blood culture positivity. After the onset of sepsis, incidence of death has been reported to increase by 7.6% every hour before appropriate treatment is initiated. Empiric antibiotics are therefore crucial for rapid treatment, but may miss the mark especially for Gram negative rods, both delaying appropriate treatment and increasing likelihood of side effects and antimicrobial resistance proliferation.

Although molecular techniques such as polymerase chain reaction (PCR) and mass spectrometry can detect the presence of certain antibiotic-resistant markers within a few hours, and PCR can work directly from positive blood cultures, the presence of these probed markers does not necessarily reflect the phenotypic resistance. Furthermore, pathogens continuously evolve to survive under antibiotic challenge. Especially true for Gram negative bacteria, this attenuates the utility of molecular (e.g. PCR) and mass spectrometric methods, the latter of which require expensive instrumentation and maintenance that are incompatible with lower resource environments.

Although phenotypic methods uniquely determine susceptibility irrespective of bacterial resistance mechanisms, their long turnaround time mandates often inappropriate empiric treatment to be administered at the earliest stage. While 48-hour post blood culture positivity) phenotypic ASTs are still the gold standard for BSIs, multiple faster clinical phenotypic alloyed standard ASTs are currently used in well-equipped hospital laboratories, including Vitek-2 (bioMerieux Inc., Durham, N.C.), MicroScan (Siemens Healthcare Diagnostics), and BD Phoenix Automated Microbiology System (BD Diagnostics). These all report AST results faster than traditional methods (e.g. BMD), but still require subculturing and isolation of pure bacteria and result in susceptibilities only after ≥30 h from blood culture positivity.

To address the need for rapid, inexpensive, and automated susceptibility determinations directly from positive blood culture, disclosed herein is an example colorimetric AST (ChroMIC) that determines minimum inhibitory concentrations (MICs) within about 5 hours of blood culture positivity. Both categorical and essential agreements on real positive blood cultures are determined relative to BMD (gold standard) and to Vitek-2 ASTs with comparable results in one-sixth the time. This inexpensive, visually or computer-determined MIC can be easily and inexpensively implemented and requires essentially no technician input after dispensing the initial sample. ChroMIC can improve patient outcomes in both high and low resource environments, while also significantly decreasing both hospital and patient cost.

In order to prepare the contrast agent, sterile human whole blood (ZenBio, Research Triangle Park, N.C.) was stored at 4° C. and used within two weeks. The sterility of purchased human whole blood was confirmed by plating on LB-agar plates and incubating at 37° C. for 24 hours. For use as contrast, whole blood was diluted in sterile cation-adjusted Mueller Hinton broth (CAMHB; BD Biosciences, San Jose, Calif.) media (10% v/v).

Relating to Gram stain and bacteria identification—once positive, Gram stains are performed through standard dye labeling and microscopic observation. Positive cultures showing Gram negative rods were selected for study if within 8 hours of turning positive. In parallel to the ChroMIC experiments, cultures were plated, colonies picked and resuspended in media for mass spectrometry-based ID (BioMerieux) and Vitek-2 based susceptibility testing.

Antimicrobial agents can include ceftazidime (RPI corp., Mount Prospect, Ill.), meropenem (Tokyo Chemical Industry, Tokyo, Japan), tobramycin (RPI corp., Mount Prospect, Ill.), levofloxacin (Alfa Aesar, Haverhill, Mass.), cefepime (Chem-Impex Int'l, Wood Dale, Ill.), gentamicin (MP Biomedicals, Solon, Ohio) and amikacin (MP Biomedicals, Solon, Ohio).

Relating to an example colorimetric AST directly from positive blood culture —BacT/ALERT FA PLUS positive blood cultures were 500-fold diluted in CAMHB media and dispensed in the 96-well plate antibiotic panels. Antibiotic panels (seven antibiotics total: ceftazidime, meropenem, tobramycin, levofloxacin, cefepime, gentamicin, and amikacin) were prepared by serial two-fold dilutions along each row of the 96-well plate. Final antibiotic concentrations ranged from approximately 0.03125 μg/mL to approximately 64 μg/mL along each row, with final dilution of blood cultures being 1000-fold. A schematic of panel layout with final antibiotics concentration is shown in Table 4. Table 4 shows a heat map showing schematic of antibiotics panel layout for both ChroMIC and BMD assays. Wells H1, H9, and H10 are media-only negative controls, while wells H11 and H12 are no-antibiotic positive controls. Wells H2 through H8 are negative controls with no sample, but amikacin, gentamicin, cefepime, levofloxacin, tobramycin, meropenem, and ceftazidime, respectively, each at 64 μg/mL. ChroMIC ASTs were performed and compared (blinded) against both Vitek-2-determined MICs and vs. BMD. Each blood culture was also plated to retroactively determine inoculum size used.

TABLE 4

| | Well Number (concentration in (μg/mL)) | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Ceftazidime (A) | 0.03125 | 0.0625 | 0.125 | 0.25 | 0.50 | 1 | 2 | 4 | 8 | 16 | 32 | 64 |
| Meropenem (B) | 0.03125 | 0.0625 | 0.125 | 0.25 | 0.50 | 1 | 2 | 4 | 8 | 16 | 32 | 64 |
| Tobramycin (C) | 0.03125 | 0.0625 | 0.125 | 0.25 | 0.50 | 1 | 2 | 4 | 8 | 16 | 32 | 64 |
| Levofloxacin (D) | 0.03125 | 0.0625 | 0.125 | 0.25 | 0.50 | 1 | 2 | 4 | 8 | 16 | 32 | 64 |
| Cefepime (E) | 0.03125 | 0.0625 | 0.125 | 0.25 | 0.50 | 1 | 2 | 4 | 8 | 16 | 32 | 64 |
| Gentamicin (F) | 0.03125 | 0.0625 | 0.125 | 0.25 | 0.50 | 1 | 2 | 4 | 8 | 16 | 32 | 64 |
| Amikacin (G) | 0.03125 | 0.0625 | 0.125 | 0.25 | 0.50 | 1 | 2 | 4 | 8 | 16 | 32 | 64 |
| Controls (H) | 0 | 64 | 64 | 64 | 64 | 64 | 64 | 64 | 0 | 0 | 0 | 0 |

Negative control wells with antibiotics and media only were allocated to ensure the sterility of media and antibiotics. Positive control wells were prepared to track bacterial growth without antibiotics and these controls were used in developing real-time automated analysis. After preparation of assays, the 96-well plates were covered with sterile sealing film (VWR International, Radnor, Pa.) and incubated at 37° C. for 18 hours with imaging as described below.

Example instruments used with the example colorimetric AST are disclosed herein. Four computer-controlled cameras (Raspberry Pi HQ) were connected to an ArduCam multicamera board in a Raspberry Pi 4B computer running the latest version of Raspian operating system. Within an incubator held at approximately 37° C., 96-well microtiter plates were held in 3D-printed holders, approximately 10 cm above the camera. A low distortion wide-angle lens was used to image microtiter plates from below, with LED illumination from above. Software collected images once every 15 minutes from each active camera over an 18-hour period. Images were analyzed both visually and by computer for red, green, and blue channel pixel intensities. Automated well detection was performed with OpenCV in python based on color value and the two most significant principal color components within each well were calculated and used to determine positive versus negative bacterial growth. Color was compared against the positive and negative control wells on each microtiter plate within each individual image and a support vector machine (SVM) was trained using the principal components of positive and negative control wells on each plate, including all wells from the first 5 images (within the first hour when no growth has occurred in any well) as additional negative control examples to account for any differences in lighting or camera conditions. SVM-derived probabilities were used to determine growth or no growth in each well. The left—most well in each row maintaining bright red color (no growth) was taken as the MIC.

Bacteria isolation and inoculum size for assays were determined by serially diluting positive blood culture in CAMHB media and plating on LB-agar. 100 μL of serially diluted samples was pipetted in LB agar (Lennox; Sigma-Aldrich, St. Louis, Mo.) and dispersed using 6 to 7 sterile rattler plating beads (Zymo Research, Irvine, Calif.). The sample dispersed LB agar plates were incubated overnight at 37° C. followed by counting colonies and estimating inoculum size (CFU/mL). Bacterial colonies recovered from this plating step were used for BMD-based MICs.

Broth microdilution of bacteria was isolated from positive blood cultures. A single colony of bacteria was inoculated in CAMHB media and incubated at 37° C. and approximately 225 rpm for about 3 hours in a MaxQ 4000 incubator shaker (Thermo Fisher Scientific, Waltham, Mass.). After incubation the sample was diluted in CAMHB media and OD600 was adjusted to approximately 0.002, and 100 μL of it was dispensed in the wells of 96-well plates containing two-fold serially diluted antibiotics. The 96-well plate were incubated at 37° C. for 18 h and the MIC was determined by visual inspection of growth (turbidity). MIC was assigned to the antibiotic concentration at which there was no visible bacterial growth. The antibiotics layout for BMD was the same as for the ChroMIC assays (e.g. Table 4).

For data analysis, categorical and essential MIC agreements were calculated for ChroMIC MICs versus those from BMD and from Vitek-2 for each antibiotic at every time point. Importantly, BMD and Vitek-2 only give a single final result, so the present faster results are compared with the standard long-term results for every time point measured to assess accuracy. Because ChroMIC measured a much wider concentration range, the inventors imposed the much narrower Vitek-2 concentration ranges on MICs for EA determinations.

Error-rates were calculated using BMD as the ground truth with minor errors (mE), major errors (ME) and very major errors (VME) defined by CLSI standards. Minor errors occur when either the test or the reference indicates intermediate resistance and the other is either sensitive or resistant. Similarly, ChroMIC ME and VME were calculated as false-resistance and false-susceptible events, respectively using BMD as the ground truth.

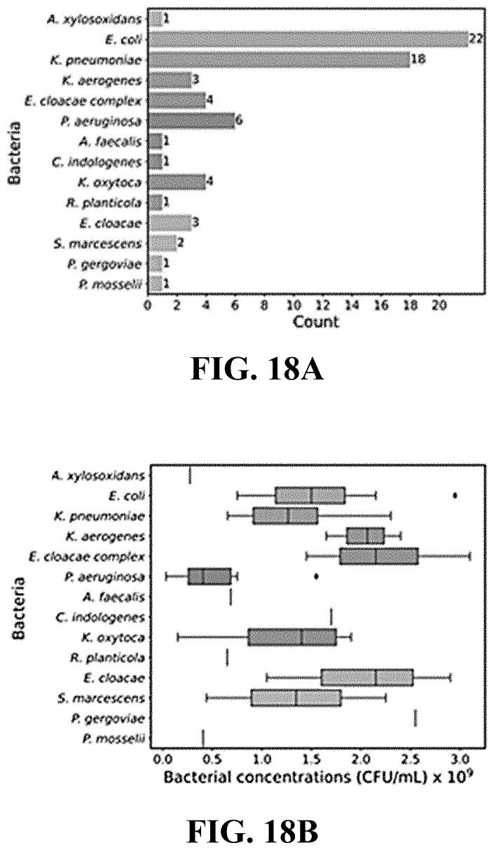

For ASTs, the standard inoculum is 5×10⁵ CFU/mL. Tested positive blood cultures had relatively consistent bacterial densities (see FIG. 18B), average 1.4×10⁹ CFU/mL), typically in the range of approximately 10⁸ to 10⁹ CFU/mL. For ChroMIC assays, all positive blood culture samples were 1000×diluted with the expectation that an inoculum 5-10×10⁵ CFU/mL would be obtained. In parallel with each ChroMIC assay, each inoculum size was determined via serial dilution and plating in LB-agar plates (see FIG. 18B). FIG. 18A shows different types of Gram-negative bacteria studied and their corresponding number. FIG. 18B shows a boxplot showing bacterial concentration (CFU/mL) in the positive blood culture of each type of Gram-negative bacteria.

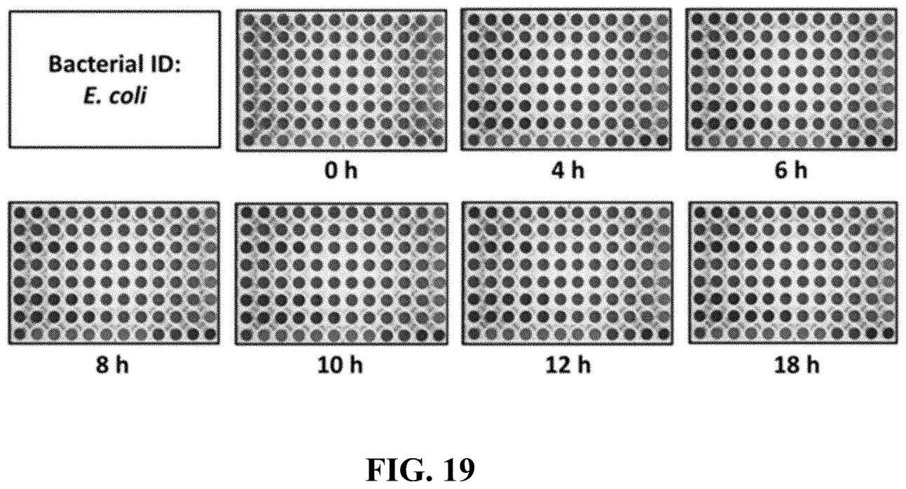

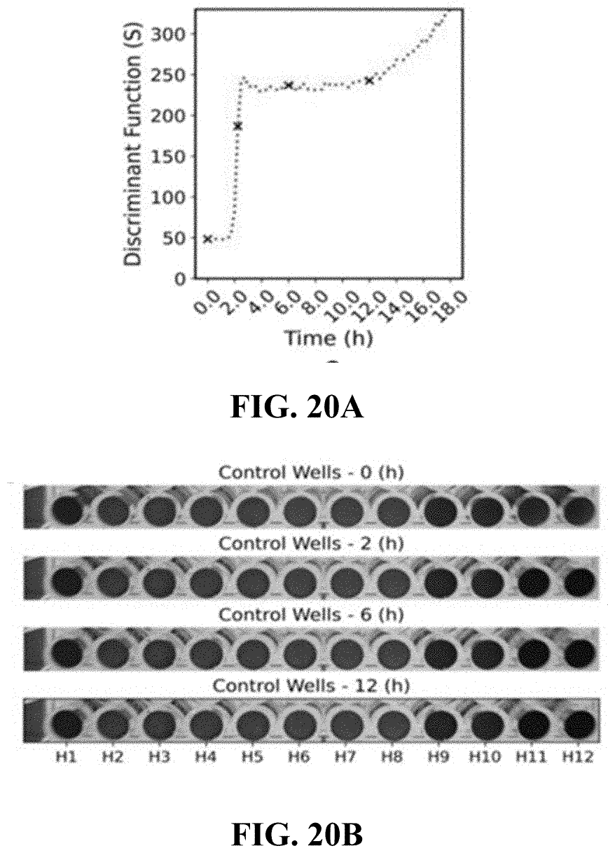

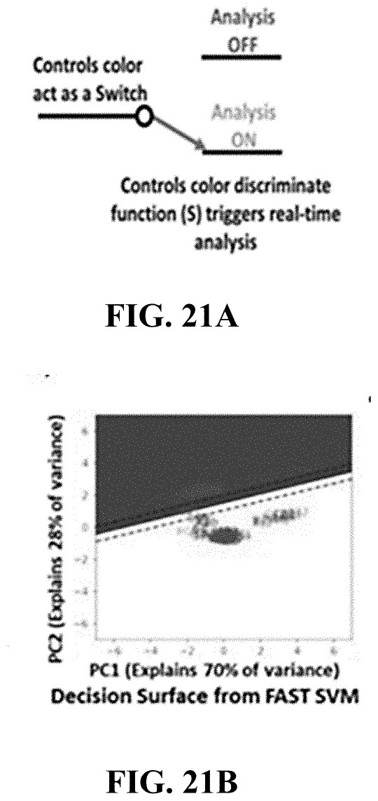

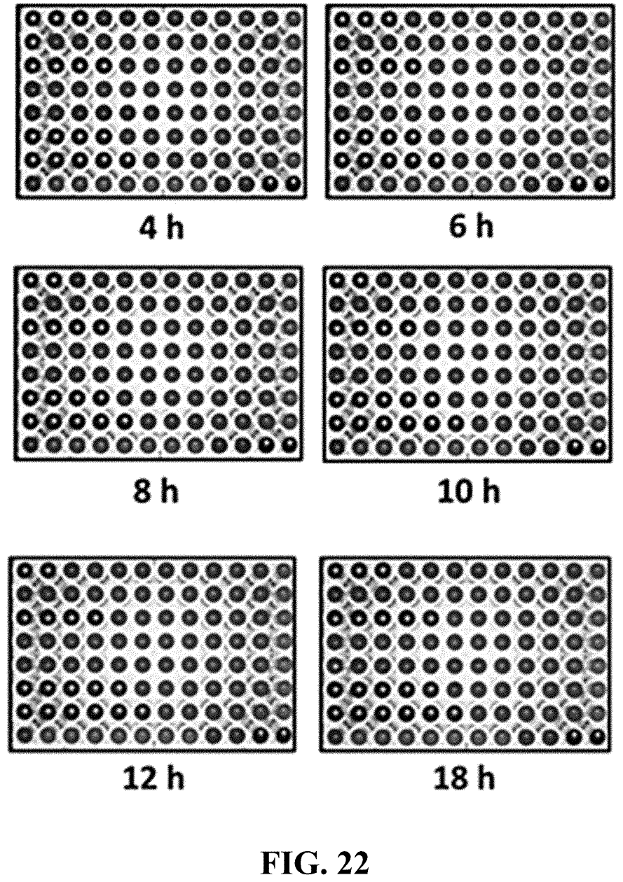

ChroMIC assays were automated to acquire one image every 15 minutes over 18 hours. MICs were determined from the image sequences as the lowest antibiotic concentration well that did not change color to dark red (growth, see FIG. 19). Wells can be automatically detected based on their color and the mean RGB color values can be taken from the average color of a 40×40 pixel area around each well centroid. Principal components using RGB values as input dimensions can be calculated for the wells in each image. MIC determination is turned on when the second principal component accounts for more than 10% of the total explained variance (i.e. when growth is observed in the positive control wells, H11, H12) and is represented by discriminant function (S), which triggers real time analysis (FIG. 21A) The S values as a function of time for this sample is shown in FIG. 20A and the corresponding control wells shown by black cross at 0 hours, 2 hours, 6 hours, and 12 hours is shown in FIG. 20B. Wells H1-H10 on each image and all wells (A1-H12) from the first hour (5 images—all before growth occurs) can be used as negative control wells. After growth is observed based on color variance, a support vector machine can be trained using all negative controls and the positive controls H11 and H12 from that plate and all plates after reporting started. Since PCs are calculated for each image, all positive and negative controls are rotated into the current image PC space. A grid search can be performed to find the optimal SVM discriminant (FIG. 20B), and probabilities are used to assign growth (+) or no growth (−) for each well. Because lower antibiotic concentrations should always show growth if a higher antibiotic concentration shows growth, the inventors used Bayesian methods to update each higher antibiotic concentration probability within a given row based on whether the adjacent lower concentration showed growth or not (FIG. 22). Extracted MICs for this sample are given in Table 5 below which shows real-time Bayesian updated MICs at multiple time-points of the corresponding images in FIG. 19.

TABLE 5

| | MIC | MIC | MIC | MIC | MIC | MIC |
|---|---|---|---|---|---|---|
| | (μg/mL) | (μg/mL) | (μg/mL) | (μg/mL) | (μg/mL) | (μg/mL) |
| Antibiotics | (4 hour) | (6 hour) | (8 hour) | (10 hour) | (12 hour) | (16 hour) |
| Ceftazidime | 0.25 | 0.25 | 0.125 | 0.125 | 0.125 | 0.125 |
| Meropenem | 0.0625 | <=0.03125 | <=0.03125 | <=0.03125 | <=0.03125 | <=0.03125 |
| Tobramycin | 0.5 | 0.5 | 0.5 | 0.5 | 1 | 1 |
| Levofloxacin | <=0.03125 | <=0.03125 | <=0.03125 | <=0.03125 | <=0.03125 | <=0.03125 |
| Cefepime | <=0.03125 | <=0.03125 | <=0.03125 | <=0.03125 | <=0.03125 | <=0.03125 |
| Gentamicin | 0.5 | 0.5 | 0.5 | 1 | 1 | 1 |
| Amikacin | 1 | 1 | 1 | 2 | 2 | 2 |

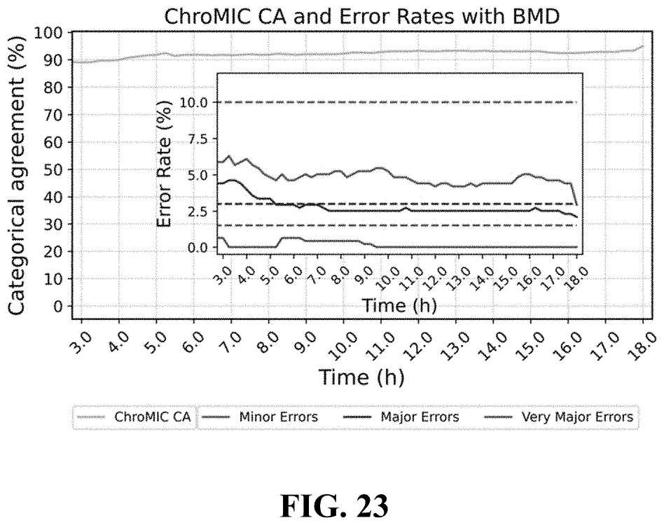

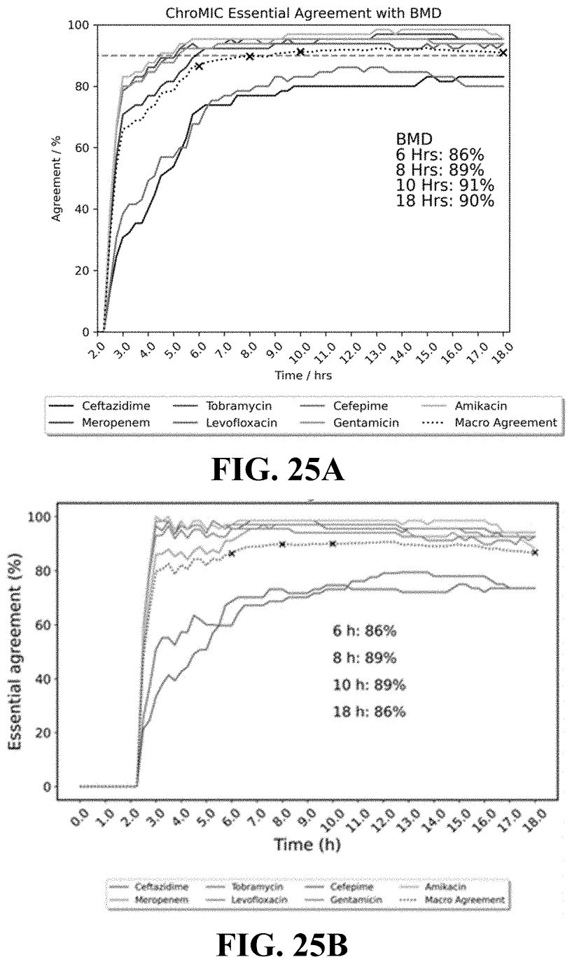

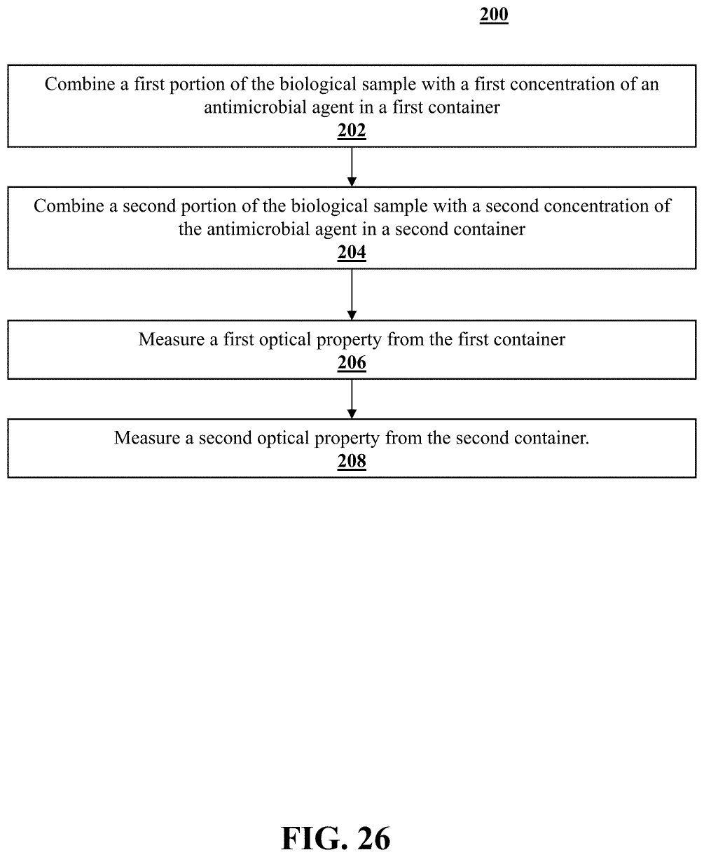

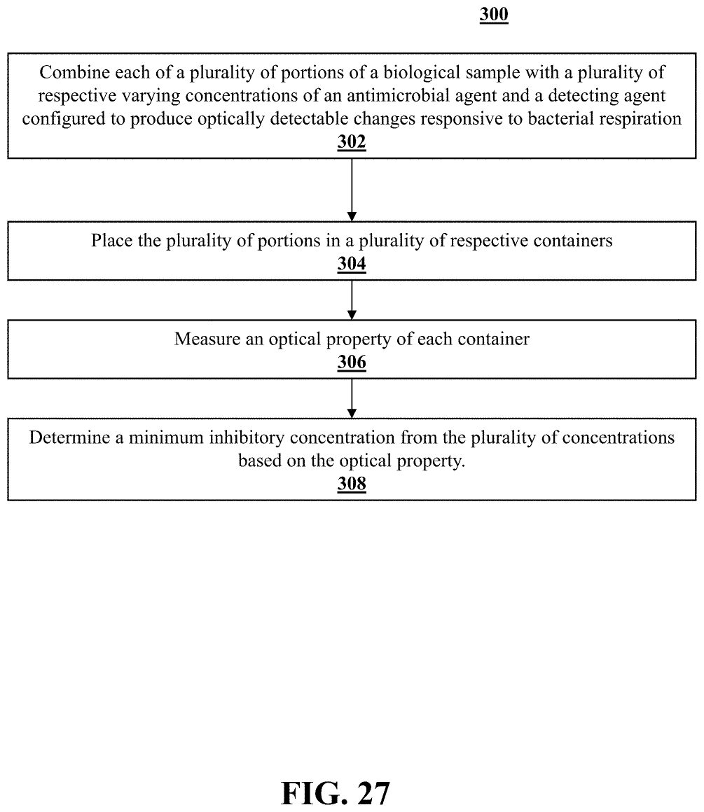

ChroMIC results were compared against Vitek-2 and BMD results without prior knowledge of either MICs or bacterial ID. Using BMD as the ground truth, ChroMIC EA was calculated over the entire tested antibiotic concentration range (0.03125 μg/mL to 64 μg/mL), and for a fairer comparison with Vitek-2 EA, ChroMIC EA was also calculated using the more limited Vitek-2 ranges when ChroMIC MICs fell within the Vitek ranges. For example, if ChroMIC reports 0.125 μg/mL for Amikacin, the inventors would adjust this to the Vitek-2 range of <=2 μg/mL and gauge whether BMD is within a factor of 2 of this adjusted ChroMIC value. Accounting for these ranges, EA with BMD exceeds 90% after 9 hours (See FIG. 25A and FIG. 25B). BMD, however, is a much longer time single measurement of susceptibility, being read after 18-24 hours. Categorical agreements with BMD shows >90% accuracy after 5 hours, with very low minor, major, and very major errors. Of the seven tested antibiotics, ceftazidime, followed by cefepime show the lowest agreement at early times. Oddly, results occasionally indicate early growth with these two antibiotics, followed by decreased MICs at the final 18-hr time point, giving a better match with BMD at long times. This is the source of the higher errors for cefepime and ceftazidime at short times, where ChroMIC occasionally over predicts the MIC. The average CA between ChroMIC and reference BMD method exceeds 90% after 4 h from blood culture positivity (FIG. 23), with minor (mE), major (ME) and very major (VME) errors all well below accepted limits for all time points (see FIG. 23). Ceftazidime and cefepime were contributors to the mE, and ceftazidime contributed most to the ME.

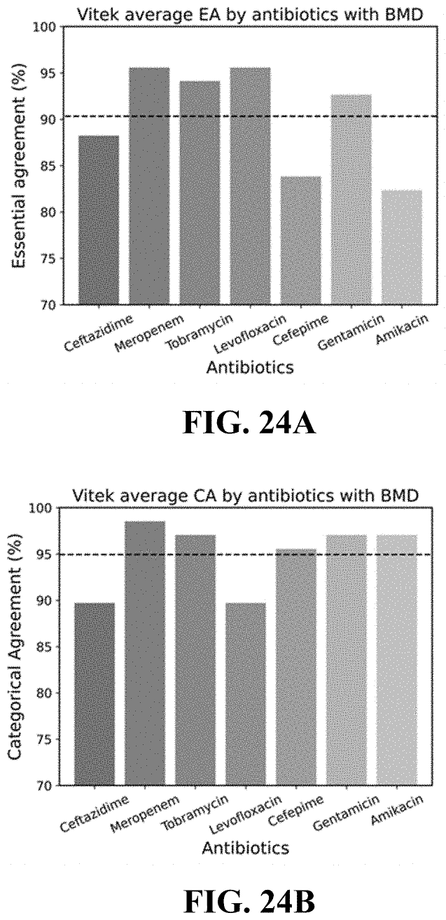

Evaluated against the clinically used bronzed standard Vitek-2, both ChroMIC EA and CA exceed 90% by ˜8 h suggesting better CA vs BMD at earlier times than Vitek-2 would be able to provide. For comparison, Vitek-2 EA and CA (using BMD as the standard) were ˜90% and 95%, respectively, when performed after subculturing and ˜10-hour AST, resulting in a delay of >24 hours relative to ChroMIC. Vitek-2, of course, only provides a single end point result (see FIG. 24A and FIG. 24B).

In conclusion, phenotypic AST remains the gold standard for determining the susceptibility of BSIs, however, long turnaround times (30 hours or greater) from positive blood culture are not suitable for targeted therapy, forcing the use of broad-spectrum antibiotics for an extended period of time. Untargeted treatment not only increases the mortality rate, especially in case of sepsis, but can also lead to an increased length of hospital stay, economic burden, side effects and AMR proliferation. Thus, multiple automated commercial systems have emerged to provide ASTs a few hours faster than conventional standard methods. However, these systems require the isolation of a colony from positive blood culture followed by AST, typically requiring around 30 hours or more following blood culture positivity.

To address the aforementioned issues, the inventors developed simple rapid automated AST/MIC assays directly from positive blood culture avoiding the lengthy colony isolation step and providing highly accurate MICs within a just few hours from blood culture positivity. Furthermore, the inventors benchmarked ChroMIC assays against the gold standard BMD for clinical blood cultures infected with Gram negative bacteria. ChroMIC assays having categorical agreement (CA) above 90%, and mE, ME, and VME values below the recommended threshold of 10%, 3%, 1.5% (at approximately 5 hours and onwards, see FIG. 23) were achieved in a low-labor, automated assay direct from positive blood culture. Faster phenotypic ASTs not only improve patient outcomes but also identify appropriate treatment regardless of resistance mechanism. The simple automated design will be of benefit to both high and low resource settings.

FIGS. 20-23 show aspects of automated real-time ChroMIC assay of an E. coli sample. An example of acquired images at different times (0 hours, 4 hours, 6 hours, 8 hours, 10 hours, 12 hours and 18 h) is shown in FIG. 19. FIG. 20A shows the discriminant function (S) as a function of time, the red dashed line shows S over time and the black cross shows S at 0 hours, 2 hours, 6 hours, and 12 hours. FIG. 20B shows color difference between positive (wells H11 and H12) and negative-controls (wells H1 to H10) over time results in change in S. FIG. 21A is a schematic showing that controls the color discriminate function triggers real-time analysis. FIG. 21B shows an exemplary Baysian-updated FAST SVM decision surface separating growth positive (dark red) and growth-negative (bright red) wells. This enables labels and MIC determinations for each antibiotic at each time point. FIG. 22 shows Bayesian updated predicted labels by FAST SVM, +sign indicates bacterial growth and −sign indicates no bacterial growth in the presence of the given antibiotic concentration in that well.

FIG. 23 shows evaluation of ChroMIC results with standard BMD. ChroMIC CA (solid orange curve) and error rates (inset) with respect to standard BMD. The dashed-red, blue, and green line represents threshold errors of 10%, 3% and 1.5% for mE, ME, and VME respectively, and the solid-red, blue, and green curves are ChroMIC mE, ME, and VME respectively.

FIGS. 24A-25 show evaluation of Vitek-2 results with standard BMD. FIG. 24A shows a bar-plot showing the average Vitek-2 EA, and FIG. 24B shows average Vitek-2 CA of each antibiotic over 65 isolates with reference BMD. The black dashed line indicates global average EA (FIG. 24A) and global average CA (FIG. 24B) of all seven antibiotics over 65 isolates.

FIG. 25A and FIG. 25B shows ChroMIC EA with respect to BMD. The dashed line indicates ChroMIC and BMD EA using the clinical Vitek-2 antibiotic range. The Macro Agreements are the average EA between ChroMIC and BMDs for the seven antibiotics and 65 isolates. FIG. 25B shows ChroMIC EA with respect to Vitek-2. The dashed line indicates ChroMIC and Vitek-2 EA. The Macro Agreements are the average EA between ChroMIC and Vitek-2 for the seven antibiotics and 65 isolates.

In summary, the AST itself is performed after the culture turns positive. This is then diluted and mixed with varying concentrations of antibiotics, then blood or hemoglobin or other contrast agents (or nothing if just using turbidity) are added, then the mixture is incubated, and real color images or spectra are recorded/analyzed. This all takes approximately 4˜5 hours and is direct from positive blood culture, without the additional plating/culturing/isolation step. Thus the AST is faster by itself and also cuts out the plating-based growth and isolation step. This makes it much faster than other approaches.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A system for detecting antimicrobial resistance of a bacteria in a biological sample, the system comprising:
a plurality of containers, each of the containers containing at least a portion of the biological sample;
a detecting agent in each of the plurality of containers, the detecting agent configured to produce optically detectable changes responsive to bacterial respiration or growth; and
an antimicrobial agent in at least a portion of the plurality of containers, wherein the antimicrobial agent is disposed in at least one of the plurality of containers.

2. The system of claim 1, wherein a first concentration of the antimicrobial agent and a first portion of the biological sample are disposed in a first container of the plurality of containers, wherein a second concentration of the antimicrobial agent and a second portion of the biological sample are disposed in a second container of the plurality of containers, wherein the first concentration of the antimicrobial agent reduces bacterial respiration in the first portion of the biological sample by a first amount, and wherein the second concentration of the antimicrobial agent reduces bacterial respiration in the second portion of the biological sample by a second amount.

3. The system of claim 2, wherein a first portion of the detecting agent is disposed in the first container and produces a first optically detectable change in the first container, and wherein a second portion of the detecting agent is disposed in the second container and produces a second optically detectable change in the second container.

4. The system of claim 2, further comprising an imaging device configured to detect the optically detectable change.

5. The system of claim 1, wherein the optically detectable changes comprise changes in color or turbidity.

6. The system of claim 1, wherein the detecting agent comprises an oxygen-sensitive chemical group.

7. The system of claim 1, wherein the detecting agent comprises a chromophore in solution.

8. The system of claim 1, wherein the detecting agent comprises a chromophore encapsulated within a carrier comprising porous hydrogel, silica, microparticles, or nanoparticles.

9. The system of claim 1, wherein the detecting agent comprises a chromophore immobilized on the surface of a carrier comprising porous hydrogel, silica, microparticles, or nanoparticles.

10. The system of claim 1, wherein the detecting agent comprises one or more of: oxyhemoglobin, hemoglobin, myoglobin, leuco-dyes, allotropes of carbon, metalloporphyrins, and oxygen sensing dyes.

11. The system of claim 1, further comprising an incubator configured to incubate the bacteria.

12. A method of detecting antimicrobial resistance in a biological sample from a subject, the method comprising:
combining a first portion of the biological sample with a first concentration of an antimicrobial agent in a first container;
combining a second portion of the biological sample with a second concentration of the antimicrobial agent in a second container;
measuring a first optical property from the first container; and
measuring a second optical property from the second container.

13. The method of claim 12, further comprising mixing a detecting agent with the sample from a subject.

14. The method of claim 13, wherein the detecting agent comprises one or more of: oxyhemoglobin, hemoglobin, myoglobin, leuco-dyes, allotropes of carbon, metalloporphyrins, and oxygen sensing dyes.

15. The method of claim 12, wherein measuring the first optical property and the second optical property comprises capturing an image of the first container and an image of the second container respectively after a passing of a time interval following combining.

16. The method of claim 15, further comprising:
comparing the first optical property and the second optical property to a control optical property from a control well comprising a third portion of biological sample; and
determining an inhibition of a bacterial growth or presence in the biological sample based on the comparing.

17. The method of claim of claim 14, wherein the detecting agent comprises an oxygen-sensitive chemical group.

18. A method for determining a minimum inhibitory concentration of an antimicrobial agent comprising:
combining each of a plurality of portions of a biological sample with a plurality of respective varying concentrations of an antimicrobial agent and a detecting agent configured to produce optically detectable changes responsive to bacterial respiration;
placing the plurality of portions in a plurality of respective containers;
measuring an optical property of each container; and
determining a minimum inhibitory concentration from the plurality of concentrations based on the optical property.

19. The method of claim 18, wherein determining the minimum inhibitory concentration comprises:
comparing the optical property of each container to an optical property measured from a control container; and
based on the comparing, determining at least one concentration at which bacterial growth is inhibited, wherein the minimum inhibitory concentration is the lowest concentration of the at least one concentration.

20. The method of claim 19, wherein measuring the optical property comprises capturing an image of the plurality of containers periodically following combining.
